(19) United States
(12) Patent Application Publication
(10) Pub. No.: US 2026/0229370 A1
(43) Pub. Date: Aug. 6, 2026

(54) QUALIQUANTITATIVE BENEFIT/RISK ASSESSMENT METHOD SUITABLE FOR THE PHYSIOLOGICAL ACTIVITY OF NATURAL MATRICES-BASED THERAPEUTIC OR BENEFICIAL PRODUCTS WITH RESPECT TO THAT OF OTHER PRODUCTS WITH PHARMACOLOGICAL OR NUTRACEUTICAL ACTIVITY BASED ON SYNTHETIC MOLECULES

(71) Applicant: Bios-Therapy, Physiological Systems for Health S.p.A., Sansepolcro (IT)

(72) Inventors: Valentino MERCATI, Sansepolcro (IT); Jacopo LUCCI, Sansepolcro (IT)

(73) Assignee: Bios-Therapy, Physiological Systems for Health S.p.A., Sansepolcro (IT)

(21) Appl. No.: 19/177,984

(22) Filed: Apr. 14, 2025

(30) Foreign Application Priority Data
Feb. 3, 2025 (EP) 25155583.5

(51) Int. Cl. G16H 70/40; G16B 25/10; G16B 40/20

(52) U.S. Cl. CPC G16H 70/40; G16B 25/10; G16B 40/20

(57) ABSTRACT

The present invention provides a new predictive in vitro or ex vivo method for defining the benefit/risk score of a therapeutic or beneficial product, a method based on data obtained from in vivo experiments for defining the benefit/risk score of a therapeutic or beneficial product as well as method for selecting therapeutic or beneficial products for further development.

### TECHNICAL FIELD OF THE INVENTION

The present invention relates to conceptual gaps in modern medicine and regulations which are focused on classic API based products rather than on new therapeutic or beneficial composition of matter consisting of 100% natural matter, having a physiological (as opposed to pharmacological) mode of action.

Modern medicine has progressively evolved toward increasing specialization, both in terms of therapeutic products—centred on isolated or synthetic active pharmaceutical ingredients (APIs)—and in terms of clinical approach, which has increasingly moved away from a holistic vision of the patient. The organism is now often interpreted through the lens of specialized subsystems (organs, functions, receptors), losing sight of systemic interconnections. This specialization has shaped not only therapeutic strategies but also the entire regulatory framework that governs therapeutic or beneficial products development and approval.

In particular, when exclusive selection and native matrices, appropriately processed through specific processes and methods, are used to create final products intended for therapeutic or adjuvating purposes, for restoring or adjuvating the organism in restoring healthy physiological states, the developers of said new products are faced with several regulatory challenges. In fact, developers of natural matrices-based therapeutic or beneficial products face significant regulatory hurdles, largely because current pharmaceutical frameworks are designed for single-compound drugs rather than complex, multi-component natural formulations. Regulatory bodies such as the U.S. Food and Drug Administration (FDA), European Medicines Agency (EMA), and other global authorities require strict characterization, reproducibility, and clinical validation-criteria that are difficult to meet for, e.g., plant-derived therapies due to their natural variability, multi-target effects, and the impossibility of defining specific therapeutic active principles. Standard drug approval pathways, such as the New Drug Application (NDA) in the U.S. or Marketing Authorization (MA) in Europe, often necessitate well-defined active ingredients, precise dosing, and clear pharmacokinetics, which do not align with the synergistic and emergent properties of plant matrices.

The entire regulatory system, including the methods for assessing the benefit/risk profile of new therapeutics, has been constructed around this reductionist model. In current practice, benefit/risk assessment is generally qualitative in nature and based on clinical trial outcomes, therapeutic efficacy and side effects observed in patient populations, and only subsequently refined through real-world post-marketing data. Notably, there is no standardized method for predictive, early-stage benefit/risk evaluation based on in vitro or ex vivo data. Nor is there an approach that interprets this data through a probabilistic and holistic model capable of generating a measurable, quantitative output, let alone diagnostic procedures capable of intercepting such parameters (e.g. thorough IVD procedures).

As a result, many plant-based products are classified under botanical drugs, dietary supplements, or traditional medicines, limiting not only their ability to claim therapeutic efficacy or receive full pharmaceutical approval but also limiting the development of innovation in this field. Additionally, one of the major gaps in the current regulatory landscape is the lack of a standardized benefit/risk assessment model tailored for multi-component natural matrices. Conventional risk evaluation is based on dose-response relationships, toxicity thresholds, and drug interaction studies, all of which are well-suited for synthetic molecules but fail to capture the holistic, synergistic effects of plant-based therapies. Conversely, the full spectrum of benefits-including immunomodulation, multi-pathway interactions, and adaptive physiological responses—can also be difficult to quantify and compare against conventional drugs under current guidelines.

This reinforces the need for new evaluation paradigms that reflect a systems-based and integrative view of human biology-something that modern regulation, shaped by increasingly compartmentalized medical thinking, has so far failed to accommodate.

Another key challenge is the requirement for Good Manufacturing Practices (GMP), ensuring batch-to-batch consistency—a major issue when dealing with natural (e.g., botanical) extracts affected by geographical, seasonal, and genetic variations. Additionally, natural matrices-based product developers also navigate intellectual property (IP) complexities. This makes securing investment for research and development difficult, as companies struggle to establish exclusive rights over their innovations. Furthermore, compliance with global safety regulations, such as REACH (Registration, Evaluation, Authorisation, and Restriction of Chemicals) in Europe or Traditional Herbal Medicine Registration in China, adds another layer of complexity, often requiring long-term toxicology studies, clinical trials, and detailed pharmacovigilance programs. These regulatory barriers significantly slow down-market entry, increase costs, and create uncertainty for innovators in the plant-based therapeutic space. On the other hand, the public is requesting more and more a drift towards naturality, under the aegis of the One Health principle (which is a principle that recognizes the interconnectedness of human health, animal health, and environmental health), therefore, requesting products for which every phase of their production falls under said principle and the use of artificial forces or substances is not permitted.

Indeed, in the field of the present invention, the therapeutic or beneficial product of interest, that is a product comprising or consisting of one or more natural matrices, maintains its natural intelligence i.e. the imprint of the domain of the living to which each constituent of the product belongs, thereby maintaining a network capable of interconnecting and recognizing itself with other networks, whether natural or artificial, that is, originally natural networks that have acquired a degree of artificiality due to interaction with artificial components. This interconnection is deemed to be fundamental to rebalance any disturbances in the network of events that are active in each interacting biological system.

Each network of each native natural matrix comprised in the product, contributes to forming the network of the final matrix of the product of interest, and can be defined as a UVCB substance (i.e. Substances of Unknown or Variable composition, Complex reaction products, or Biological materials) according to the REACH (Registration, Evaluation, Authorisation, and Restriction of Chemicals) definition since, being a product processed respecting its self-assembly peculiarities, it cannot be determined nor validated on the basis of small molecule chemistry protocols.

Each network is characterized by the establishment of connections within the matrices in the final product and within the physiological actions exerted by the product on the receiving organism. The production validation of this kind of products can be carried out and confirmed using probabilistic models based on the link between the conservation of a physiological activity profile and descriptors of the matrix per se generated using multiple bio-physical analytical systems, including spectroscopy (NIR and other techniques), mass spectrometry, and paper or X-ray crystallography (fractal measurements) as disclosed in Japanese patent 7616724 and in PCT/IB2024/054526. Indeed, although useful, the conventional molecular chemical definitions of individual substances contained in matter cannot be used for validating this kind of products as they are not representative of the overall effectiveness and quality thereof.

The selection of matrices intended for administration must be validated according to the updated and specific current taxonomic criteria for the animal, plant and mineral kingdoms. In case of use in combination with natural physical phenomena, it will be necessary to validate the relationship between action and effectiveness on a case-by-case basis, considering sound effects (music or other forms) and those in the field of wave-particles, including those of a quantum nature.

In the current state of the art, it will not always be possible to outline a fully described mechanism of action; however, it will be possible to validate the action and reaction in the interconnection of the respective networks, already validated at a biophysical level.

The invention aims to allow the selection and provision to the persons in need thereof, of new therapeutical or beneficial entities or products, as well as systems capable of rebalancing, activating, or limiting physiological functions in specific metabolic states of living organisms, that are always in continuous transformation.

The preparations conceived in this way can rebalance the psycho-neuro-endocrine-immunological system, considered as a single system that governs and manages all the other systems.

The invention contributes to a new state of the art that goes beyond the alchemical technologies in the medical field, the beginning of which can be traced back to the early years of the sixteenth century, bringing products and processes back to the conceptual One Health objective already mentioned. The invention proposes a new declination of artificial technologies and of those that naturally self-assemble matter, recognizing existing rules or finding new ones in order to guarantee the constitution of entities that can be validated, mainly based on the concept of validating of their effect and activity on other organisms. The latter, being living beings in continuous transformation, require an evaluation of their physiological state within defined intervals, which is a concept that today falls within the personalized medicine. The present invention fits into the concept of scientificity, understood as the set of knowledge that can demonstrably validate the effects of theoretical modes of action. Today, these methods find application in the establishment of interconnections between all forms of life, in a context in which technological innovations advance at such a pace as to risk compromising the interconnection between human-generated (artificial) intelligence and natural.

Since the activities of therapeutical or beneficial natural matrix-based products are not currently covered by the state of the art, it will be necessary to consider the entire product cycle, from the end user to the social context concerned, under the concept of One Health.

The operational paradigm within which the invention was prepared has been herein denominated as “Bios Physiological Health”.

This paradigm aims to introduce into the field of medical art an innovative approach for the treatment and self-management of health using natural matrices, alone or in combination, in order to rebalance the normal physiological states of various living entities, including humans, through endogenous physiological actions triggered by the product. It is a matter of identifying, selecting, and assembling natural entities which possess emerging properties, validable through the final product's physiological mode of an action and other methods evolved in recent decades.

A reading of the context according to both technical-scientific and humanistic canons, integrated in their transversality, constitutes the foundation of the proposed invention. Although some of the properties of each matrix part of the product may already be known the emerging properties of the new composition are unexpected.

Of particular relevance is the role of determining the genetic and epigenetic aspects determining the network representing natural matrices and their description at the level of their specific isotopic abundance.

In order to fulfil the Bios Physiological Health paradigm, each phase of processing, from the selection of the reproductive material to the agricultural and industrial phases, up to the methods of use, must preserve as much as possible the integrity of the native programming heritage, inserted into the natural intelligence of each entity of creation, at least as far as known in our terrestrial dimension. It will be essential to validate matrices coming from epigenetic realities similar to the reference one, recognized as a Reference Standard for its specific emerging properties on the metabolism of other living beings, including humans. By way of example, one of the factors that negatively influences epigenetic differentiations is represented by different soil conditions, together with circadian, monthly, and annual variations. To preserve the properties of the natural system, which are the only ones that can claim physiological interconnection with the whole of creation, it is not possible to use substances derived from alchemical processes, such as distillation, other processes of synthesis or hemi synthesis, or products derived from genetic modifications or genetically modified organisms. A new interpretation of the mysteries of natural programming, responsible for the vital evolution of organic and inorganic matter, is needed. The consolidation of scientific evolution in recent decades allows us to reposition the understanding of the genesis of a progress based on reductionist determinism, founded on the development of alchemical processes starting from the beginning of the 16th century, which in medicine, with Paracelsus, marked the beginning of the current evolutionary process, known as the Anthropocene.

The term Anthropocene describes the current phase of human evolution and can be dated back to different eras. If considered in the context of this invention, the key date can only be 1492, which represents the end of the humanistic/neo platonic period of the early Renaissance. This period was represented politically by Cosimo the Elder and Lorenzo de'Medici, with artists and scientists such as Piero della Francesca, Luca Pacioli, Leonardo and Dürer. In the 16th century, alchemical research seen as a human possibility of dominion over nature, has evolved until today under the aegis of artificial intelligence as opposed to the natural one, inspired from the biblical thought according to which “man will dominate over all creation”, with the aim of improving divine creation.

1492 is a symbolic date: in that year Lorenzo de'Medici and Piero della Francesca died, while Columbus discovered America. The human species abandoned the fifteenth-century Neoplatonic path to follow the Judeo-Catholic one, where the alchemical practices of Paracelsus applied to medicine marked the transition to the Renaissance mannerism of the 1500s, which, up to the present day, has led us towards a full-blown and irreversible sixth extinction.

The present invention, with the demonstration of feasibility of the resulting industrial discoveries in the medical field, but in principle adaptable to any production field, intends to address the change in evolutionary paradigms. We often talk about defending biodiversity without ever addressing the real problem, guiltily obscured, of the billions of tons of exogenous and non-biodegradable artificial substances released into the planetary system, with the certainty of irreversibly poisoning the sources of life, while the “carpe diem” approach prevails over the survival instinct of the species.

The invention is presented primarily in the patent context, with the hope of opening new areas of research that explore and share natural intelligence, rather than artificial intelligence, which, can do very little to stop or slow down the sixth extinction, or to lay the foundations of an alternative progress to the current one. The inventor Valentino Mercati, together with his collaborator Jacopo Lucci, has undertaken the path of researching in nature itself what can be useful to the living systems, and has developed a knowledge in the agricultural and industrial production system for over 40 years, presenting numerous patent applications following this operational strategy. The patents filed in the past relating to the present inventive process are essentially based on instrumental and diagnostic readings based on principles related to chemistry for the connection of physiological actions with the emerging properties of natural matrices and the innate defences of each individual living being with which they interconnect.

The analyses that followed and inspired the approach herein disclosed were unthinkable just a few decades ago, due to the technological impossibility of reading the genetic and epigenetic information written in the cells of every living organism, and the role of atomic isotopic differentiations in the molecular self-assembly and interconnections of every single unity/individuality with the “universe”. The conceptual difficulty in moving from the reassuring management parameters of the artificiality of molecules—at least partly purified and linked by powerful thermodynamic forces that allow strong bonds such as covalent ones, which acts on reduced molecular scopes of other organisms—to natural matrices, mysterious by definition and, still viewed today as part therapeutically unreliable, is extremely high.

If a new inventive interpretation is needed for a new medical state of the art after five centuries of alchemical reductionism, this interpretation must connect the most distant concepts and processes in a single application field. This is due, as already expressed, to a philosophical legacy that questions the human condition: the human species was created like all the others by the original vital intelligence with the purpose of life itself, as far as we can assume, to dominate on creation, or has it been experimentally endowed with different faculties from other organisms, already favourably inserted into creation, to constitute a new ecological niche at the service of the universe? The answer to this dilemma does not arise for the current invention: humanity will have to return to the Neoplatonic thought of the early Renaissance, and the experimental duality of the human species must emancipate itself from the mindset of dominance in order to share its unique faculties within the universe with all of creation. Humanity will need to reconsider the warning of Leonardo da Vinci: “Man can only create his own offspring . . . ” and reflect on the melancholic thoughts of wise figures like Piero della Francesca, Luca Pacioli, and Dürer, regarding the impossibility of understanding and representing the beauty of creation and deciphering its mysteries.

The era has arrived for the acquisition of new research centres in molecular and cellular biology, with an indispensable focus on bioinformatics and the new physical sciences. Today, the inventor can base research strategies and socioeconomic applications in new therapeutic fields, particularly those that are complex and/or chronic-degenerative, where the restoration of metabolic balance for organisms either naturally or artificially disturbed will become an integral part of a future that is already present.

The present invention redefines the medical state of the art by introducing a holistic evaluation model that aligns with personalized medicine and systems biology. Unlike reductionist pharmacological approaches that isolate active ingredients, this invention acknowledges the self-organizing complexity of natural systems.

By establishing a rigorous scientific basis for the assessment of natural matrix-based therapies, this invention fills a critical gap in current medical research. It responds to the limitations of a medical and regulatory framework built around highly specialized, single-compound pharmacology, which lacks tools for evaluating complex, multi-target products in a systemic way. It offers a new paradigm for benefit/risk evaluation, one that integrates bio-physical analytics, probabilistic modelling, and physiological impact assessment introducing, for the first time, a quantitative and predictive method based on in vitro or ex vivo data, capable of supporting early-stage decisions and restoring scientific validity to holistic approaches. This ensures that natural intelligence, rather than artificial intelligence, guides future advancements in medicine.

### BACKGROUND OF THE INVENTION

According to the current procedures for assessing Benefit/Risk Scores in medical products, regulatory agencies such as the U.S. Food and Drug Administration (FDA), the European Medicines Agency (EMA), and the World Health Organization (WHO) employ rigorous benefit/risk assessment frameworks to evaluate medical products before granting market authorization. These assessments rely on quantitative and qualitative methodologies to weigh a product's therapeutic benefits against its potential risks, ensuring that the overall impact on public health is favourable and justifiable.

In addition to regulatory approval, many countries also require Health Technology Assessment (HTA) as part of the decision-making process for pricing, reimbursement, and market access. HTA is a multidisciplinary evaluation framework that considers not only clinical efficacy and safety but also economic, social, and ethical implications of a medical product.

Standard benefit/risk assessment methods typically include:

Clinical Trial Data Analysis: Evaluating safety and efficacy through randomized controlled trials (RCTs), which provide statistical comparisons of the investigational product against a placebo or existing standard treatments.

Pharmacokinetics and Pharmacodynamics (PK/PD) Modelling: Measuring how a drug is absorbed, distributed, metabolized, and excreted, along with its biological effects at different concentrations.

Toxicology and Safety Profiling: Identifying potential adverse effects, contraindications, and drug interactions through preclinical (animal and in vitro) and clinical (human trials) studies.

Structured Decision-Making Approaches: Using models such as multi-criteria decision analysis (MCDA) and quantitative benefit-risk models (QBRMs) to balance benefits and risks systematically.

Standardized Risk Management Plans (RMPs) and Post-Market Surveillance: Ensuring continuous monitoring of safety signals through pharmacovigilance programs.

Health Economics & Cost-Effectiveness Analysis (for HTA): Assessing whether a treatment provides sufficient value for its cost by comparing quality-adjusted life years (QALYs) and other economic indicators.

Risk Management Plans (RMPs) and Post-Market Surveillance: Ensuring continuous monitoring of safety signals through pharmacovigilance programs.

These methodologies are highly structured and reproducible, making them suitable for synthetic drugs and well-defined biologics, where individual active pharmaceutical ingredients (APIs) can be isolated, characterized, and dosed with precision.

These procedures cannot be directly applied to natural matrices-based therapeutic or beneficial products; in fact, despite their effectiveness in conventional pharmaceuticals, these existing benefit/risk and HTA assessment procedures face critical limitations when applied to natural matrix-based therapeutic or beneficial products, primarily due to the inherent complexity and variability of natural substances.

Unlike conventional drugs that have a single, well-defined active compound, natural matrices contain multiple bioactive compounds that interact with each other as a network system, and contribute to their therapeutic effect synergistically, therefore, they lack a single active ingredient. As modern medicine, also modern regulatory science is reductionist as it focuses on identifying a single molecular entity responsible for efficacy, whereas natural matrices-based products exert their effects through complex, multi-target interactions. Current clinical and pharmacological models are not designed to capture or quantify these emergent properties, making standard PK/PD modelling insufficient. In addition, natural matrices are inherently subject to biological, environmental, and genetic variations, leading to batch-to-batch differences in composition.

Pharmaceutical regulations and HTA models require highly standardized compositions with precise dosing, something that natural matrix-based products cannot easily provide without altering their intrinsic properties. Current analytical techniques struggle to measure efficacy without reliance on a single molecular marker, making regulatory and HTA approval difficult. Natural matrices often contain hundreds of chemical constituents, some of which may have non-linear dose-response relationships or adaptive physiological effects that are difficult to predict using standard toxicology models. Traditional safety studies are designed for synthetic compounds with well-defined pharmacokinetics, making it hard to assess the risk of natural matrices where multiple components interact dynamically with the body's metabolism.

Due to the fact that natural matrices-based products do not follow a linear dose-response curve and their benefits may be long-term and preventive, whereas cost-effectiveness models prioritize immediate, measurable outcomes it is difficult to quantify efficacy in economic terms.

Substantially, there is in the art, the need for a new Benefit/Risk and HTA assessment paradigm to bridge the gap between regulatory and HTA requirements and the reality of natural matrix-based therapeutics providing a new benefit/risk and health technology assessment model.

Until such a technology is developed and accepted by regulatory agencies and HTA bodies, natural matrix-based therapeutics will remain at a disadvantage, as they cannot be adequately evaluated using current pharmaceutical benefit/risk and HTA models. Ultimately, the present invention lays the groundwork for a new paradigm in therapeutic development—one that not only aligns with the principles of One Health and natural intelligence, but also introduces a forward-looking model for benefit/risk assessment.

By enabling a quantitative, early-stage, and probabilistic evaluation of both beneficial and potentially adverse physiological effects—derived from in vitro or ex vivo transcriptional data—the invention addresses a fundamental limitation of current regulatory and scientific practices. It offers a concrete, measurable alternative to the prevailing qualitative methods designed around single-compound synthetic drugs, thus restoring scientific dignity and regulatory feasibility to complex natural products.

This approach does not merely complement the current system—it redefines it, offering a path toward safer, more effective, and truly integrative medicine.

### SUMMARY OF THE INVENTION

The present invention relates to a novel approach to quantify differences in the benefit/risk profiles of therapeutic or beneficial products for a given pathological area of interest. The approach takes into account transcriptional effects assessed in an in vitro or ex vivo context. It introduces an analytical method designed as exploratory tool suitable for early research and development (R&D) phases.

The method is based on an integrated approach utilizing transcriptomic data and advanced biological pathway analysis tools (such as Ingenuity Pathway Analysis e.g., IPA), to identify gene expression changes and the resulting modification of relevant biological activities, addressing both therapeutic efficacy and potential adverse effects.

Specifically, the transcriptional profiles generated are analysed to determine differentially expressed genes (DEGs) and key biofunctions linked to an action capable of counteracting the pathologies of interest and specific side effects caused by the standard reference drug for each treatment thereby allowing calculation of the benefit/risk score of different therapeutic or beneficial products based on a conceptual integrated approach of transcriptional profiles obtained from in vitro or ex vivo studies. The method of the invention is particularly suitable for therapeutic or beneficial products comprising natural matrices. Benefit/risk ratio is a crucial parameter extremely helpful in terms of appreciation of the differences between such key features of therapeutic or beneficial options. Nevertheless, such a parameter is currently hard to be reassumed into an objectively calculated single numerical parameter that could facilitate a first approach to the comparison between therapeutic solutions. Here we provide a general method capable of correctly predicting and objectivate the comparison between the risk/benefit ratio of a new therapeutic or beneficial product of interest for the treatment of given a pathology or pathological state vs. the known reference drug for the treatment of the same pathology or pathological state, the method of the invention can be applied even based exclusively on in vitro or ex vivo transcriptomics data.

Hence, an object of the invention is a computer implemented method for providing a benefit/risk score of a therapeutical product of interest based on transcriptional in vitro or ex vivo data comprising the following steps
1. performing a transcriptomics analysis by
1.1 providing samples of a biological substrate representing the pathology or pathological state treated by said product and
a. treating one or more of said samples, hereinafter samples a, with said product;
b. treating one or more of said samples, hereinafter samples b, with a reference drug for the treatment of said pathology or pathological state; and
c. using one or more samples, hereinafter samples c, of said biological substrate as relevant control;
1.2 extracting RNA from each of said samples a. b. and c.
1.3 performing a transcriptome raw data analysis from the RNAs extracted in 1.2 and identifying the differentially expressed genes (DEGs), in each of samples a and b, with respect to said samples c, and their expression fold changes with respect to said samples c thereby obtaining fold changes values for each of said DEGs;
1.4 performing a pathway enrichment and functional analysis of transcriptomic data on the list obtained in 1.3 thereby obtaining numerical values representing the variation in terms of magnitude and directionality (i.e., upregulated or downregulated) of biological activities related to the differential expression of said DEGs in each of samples a and b normalised with respect to samples c;
2. determining the benefit score by
2.1 selecting, among the biological activities of 1.4, the biological activities which are relevant to the therapeutic indications of said product,
2.2 converting the numerical values obtained in 1.4 of said relevant biological activities into absolute values,
2.3 summing said absolute values of each of said biological activities thereby obtaining the benefit score of the product under examination,
3. determining the risk score by
3.1 providing a list of the known side effects associated with a reference drug for the treatment of said pathology
3.2 determining, by pathway and functional analysis, the biological activities associated with said side effects,
3.3 building a risk in-silico model using pathway and functional analysis by establishing a relationship between gene expression pattern obtained at point 1.3 and the biological activities determined at point 3.2, for samples a and b;
3.4 inputting the relevant fold changes values for each DEGs of samples a. and b. obtained in 1.3 to the risk in-silico model obtained at point 3.3. and using pathway and functional analysis to obtain data representing the direction and the magnitude of the regulation of each biological activity determined at point 3.2 and transforming said data into corresponding numerical values;
3.5 summing each positive value of said numerical values obtained in 3.4 thereby obtaining a final value representing the risk score of the product under examination, wherein, when said final value is <than a predefined positive minimal value, it is automatically corrected to said positive minimal value,
4. providing a benefit/risk score value of the product of interest as the ratio between the benefit score value obtained at point 2.3 and the risk score value obtained at point 3.5 Another object of the invention is a method for the selection of one or more new therapeutic or beneficial products for clinical development wherein the benefit/risk score for one or more therapeutic or beneficial product of interest for the treatment of a given pathology or pathological state and for the reference drug for the treatment of said pathology or pathological state are provided performing the steps defined in the description and in the claims (see method above) wherein, when at least one said benefit/risk score value provided for said therapeutic or beneficial product of interest is equal to or higher than said benefit/risk score value provided for the reference drug, each of said at least one said therapeutic or beneficial product of interest is selected for clinical development.

A further object of the invention is a computer-implemented method for producing a therapeutic or beneficial product, comprising determining its benefit/risk score based on transcriptional in vitro or ex vivo data, the method comprising the following steps:
1. performing a transcriptomics analysis by
1.1 providing samples of a biological substrate representing the pathology or pathological state treated by said product and
a. treating one or more of said samples, hereinafter samples a, with said product;
b. treating one or more of said samples, hereinafter samples b, with a reference drug for the treatment of said pathology or pathological state; and
c. using one or more samples, hereinafter samples c, of said biological substrate as relevant control;
1.2 extracting RNA from each of said samples a. b. and c.
1.3 performing a transcriptome raw data analysis from the RNAs extracted in 1.2 and identifying the differentially expressed genes (DEGs), in each of samples a and b, with respect to said samples c, and their expression fold changes with respect to said samples c thereby obtaining fold changes values for each of said DEGs;
1.4 performing a pathway enrichment and functional analysis of transcriptomic data on the list obtained in 1.3 thereby obtaining numerical values representing the variation in terms of magnitude and directionality (i.e., upregulated or downregulated) of biological activities related to the differential expression of said DEGs in each of samples a and b normalised with respect to samples c;
2. determining the benefit score by
2.1 selecting, among the biological activities of 1.4, the biological activities which are relevant to the therapeutic indications of said product,
2.2 converting the numerical values obtained in 1.4 of said relevant biological activities into absolute values,
2.3 summing said absolute values of each of said biological activities thereby obtaining the benefit score of the product under examination,
3. determining the risk score by
3.1 providing a list of the known side effects associated with a reference drug for the treatment of said pathology
3.2 determining, by pathway and functional analysis, the biological activities associated with said side effects,
3.3 building a risk in-silico model using pathway and functional analysis by establishing a relationship between gene expression pattern obtained at point 1.3 and the biological activities determined at point 3.2, for samples a and b;
3.4 inputting the relevant fold changes values for each DEGs of samples a. and b. obtained
in 1.3 to the risk in-silico model obtained at point 3.3. and using pathway and functional analysis to obtain data representing the direction and the magnitude of the regulation of each biological activity determined at point 3.2 and transforming said data into corresponding numerical values;
3.5 summing each positive value of said numerical values obtained in 3.4 thereby obtaining a final value representing the risk score of the product under examination, wherein, when said final value is <than a predefined positive minimal value, it is automatically corrected to said positive minimal value,

The methods of the invention are advantageously applicable to natural matrices-based therapeutic or beneficial products, that cannot be defined in terms of single active ingredients with measurable dose-response relationships, as demonstrated in the description and in the examples.

### Glossary

Unless otherwise defined herein, scientific, and technical terms used in connection with the present invention shall have the meanings that are commonly understood by those of ordinary skill in the art. Further, unless otherwise required by context, singular terms shall include pluralities and plural terms shall include the singular.

In any point of the present specification or of the claims, the expression “comprising” or “comprise(s)” can be replaced by “consisting of” or “consist(s) of”.

A Benefit/Risk evaluation is, in the state of the art, merely a qualitative measure used to evaluate the overall value of a beneficial or medical product, intervention, or treatment by comparing its therapeutic or beneficial benefits against its potential risks or harms. This score is crucial in regulatory decision-making, health technology assessment (HTA), and clinical practice, helping determine whether a treatment provides a favourable balance between its positive effects and possible adverse outcomes. The present description provides a Benefit/Risk score therefore, the term relates to a qualitative and quantitative measure.

A “natural matrix” in the present application refers to a material consisting of a network represented by a broad number of components/constituents obtained (e.g. extracted) directly from a member of the natural kingdom or a naturally occurring portion thereof (i.e., from a natural raw source), without significant processing or synthetic alteration, wherein “without significant processing or synthetic alteration” is intended that no denaturing processes are used for obtaining the matrix from the raw source. In other words, the natural raw source is processed only by manual, mechanical or gravitational means e.g. by dissolution in water or other naturally occurring solvents, such as water, water-alcohol solutions etc.; by flotation; by extraction with water or other naturally occurring solvents; by steam distillation or by heating solely to remove water or any other naturally occurring solvent; or extracted from air by any means and with the provision that “natural matrix” excludes said member of the natural kingdom “as such” i.e. non-processed. In particular, according to the invention, a natural matrix is a 100% natural and biodegradable material, consisting of natural components that have not been denatured by the process for the production of the matrix from the starting raw materials without intentional addition of synthetic products along the whole process. In the present description, 100% biodegradable is considered as “readily biodegradable” according to an OECD biodegradability test. These features guarantee the maintenance of the matrix effect which is conferred to the matrix by the presence of structural interactions by its components (material interactions) and functional interactions that become evident upon exposure of a biological system to the natural matrix (immaterial interactions). In other words, a natural matrix, or a mixture of natural matrices, are materials obtained from entities that are self-assembled in nature and processed so to preserve their native bio-physical characteristics which determine their physiological interaction with other living organisms, such as the human organism. Their emerging properties can be expressed by contributing to the rebalancing of metabolic processes or states of the receiving organism and/or of some organs or tissues alongside the physiological actions that will be activated in each specific context. According to the present invention the natural matrix can be from a material obtained from any source in the life kingdoms i.e., Monera, Protista, Fungi, Plantae and Animalia. The term hence encompasses a plant natural matrix, an animal natural matrix, a fungi natural matrix, a Protista (archaea or bacteria) natural matrix, a Monera natural matrix. A natural matrix may also comprise natural inorganic materials such as minerals obtained from natural raw materials. A synonym of natural matrix or one or more natural matrices in the present description is “complex natural system” or “natural material” as defined below.

An example of naturally occurring portion of an organism may be represented by e.g., roots, leaves, bark, fruit, flower, of a plant or sections thereof, organs, tissues.

In any part of the description the general term natural matrix can be substituted with:
a plant natural matrix or a natural matrix obtained from a plant,
an animal natural matrix or a natural matrix obtained from an animal or from an animal product such as eggs or milk,
a fungi natural matrix or a natural matrix obtained from a fungus,
a Protista natural matrix or a natural matrix obtained from a Protista,
a Monera natural matrix or a natural matrix obtained from a Monera,
or with a plant material and/or extract, an extract from an animal tissue or organ, fungi and/or a fungi extract, or a mixture thereof wherein the extraction process does not encompass denaturing steps (e.g., temperature or the use of denaturing solvents).

Plant is synonymous with herb.

The term “natural” matrix emphasizes the retaining the integrity and complexity of networks of constituents/components as in the original natural source due to the absence of denaturing treatments for the obtainment thereof. A natural matrix hence does not encompass compositions of natural origin that are enriched in specific molecules of artificial synthesis or isolated from a natural raw material. In addition, a natural matrix is obtainable only with processes that do not act through extensive processing or chemical modification, isolation, purification, or molecular extraction.

Due to the supramolecular self-assembly of the constituents/components of a natural matrix and the presence of functional interactions among them, the whole matrix behaves as a complex network that does not interact with a single target molecule but that interacts with a network of recipients (also organised as a network) in the receiving organism. Therefore, the interaction natural matrix-receiving organism is not, as for common pharmaceutical APIs the result of a point-to-point interaction, but the result of an “interactor” networks (i.e., the matrix)-“receiver” network (i.e., the organism to whom the matrix is administered) interaction.

The term natural matrix can be also substituted in any part of the description and claims with complex natural system.

Nowhere in the description and in the claims the term natural matrix can be interpreted as “a product of nature” as such, rather, a natural matrix is a product obtained from a natural organism and processed (e.g. extracted) therefrom by techniques that do not substantially alter biological structure and the relevant supramolecular and functional interconnections among the components within the matrix as mentioned above, i.e., without the use of denaturing techniques and that does not comprise additional isolated or synthesised molecules or classes of molecules.

Emerging properties according to the present description and to the art, the term defines the properties of a natural matrix or of a natural material according to the present specification, i.e., properties that are not represented by the mere sum of properties of each singled out constituent/component of said matrix/material but by the both functional and structural interactions among all constituents/components of the matrix/material that are also the result of the supramolecular self-assembly of said components/constituents within the matrix/material itself.

“Emerging properties” hence refer to technical effects, such as therapeutic or homeostasis—adjuvating properties (i.e., beneficial effect), that the interactions and relationships among the constituents/components of a natural matrix exert on a receiving living system. By definition, emergent properties are properties that are not immediately evident or even predictable based solely on the individual characteristics of each constituent/component of the matrix. Instead, they “emerge” when all the constituents/components of the matrix networks interact with one another and with the living system receiving network in a dynamic and complex way. Emerging properties have been broadly discussed in the art in various scientific and systems-oriented fields, including physics, chemistry, biology, and complex systems theory.

Emerging properties are hence properties that cannot be predicted a priori by the quali-quantitative knowledge of each component of a given composition or matrix and that, consequently, cannot be ascribed to one or more specific API. Hence, although multidrug compositions can show unpredicted synergic effects, the properties of said compositions are still ascribed to the specific APIs and quantities thereof contained therein.

In the case of emerging properties, characteristic of natural matrices, the observed emerging properties cannot be reconducted to specific APIs and are maintained in different batches of a given matrix or a given mixture of matrices notwithstanding the different quali-quantitative composition of said batches (functional resilience see below).

Synthetic according to the present description has the meaning conventionally accepted in chemistry. Conventionally, in chemistry, the term “synthetic” refers to the origin or source of a material or substance. Synthetic substances or materials are produced by man through artificial synthesis i.e., through laboratory chemical reactions usually by reacting simpler chemicals to create more complex ones through processes that often use different pathways, temperature conditions, pressure conditions, energy sources and/or catalysers from those used by living organisms.

Examples: Synthetic substances or materials include plastics, pharmaceutical drugs, and many industrial chemicals. For example, nylon is a synthetic polymer made through chemical synthesis, and aspirin is a synthetic drug produced through specific chemical reactions.

Hallmark of a disease or of a pathological or medical condition according to the present description has the meaning conventionally used in the art. They can be readily identified by the skilled person in specialised databases, through pathway and function databases, or through disease specific databases. Hallmarks of a disease are known to be indicators that can mark the progression or control of a given disease or pathological or pre-pathological condition and taken together are usually representative of the general pathological state associated to a given pathology. These hallmarks (also called ‘key indicators’) are typically a set of features or patterns that a physician would monitor, over time, to track the onset, the progression or regression of a particular illness. In summary, a hallmark of a disease is a defining feature or characteristic whose modification is indicative of a given pre-medical or medical condition, aiding in its identification, diagnosis, monitoring and understanding. By way of example, for neurodegenerative diseases (NDDs) at least the following eight hallmarks of NDD are known in the art: (pathological protein) aggregation, synaptic and neuronal network (dysfunction), (aberrant) proteostasis, cytoskeleton (abnormalities), (altered) energy homeostasis, DNA and RNA (defects), inflammation (increase), and neuronal cell death (increase). In cancer research, the hallmarks of cancer are a set of distinctive characteristics that are commonly found in cancer cells. These hallmarks include (sustained) proliferative signalling, (evasion of) growth suppressors, (resistance to) cell death, (enabling) replicative immortality, (inducing) angiogenesis, and (activating) invasion and metastasis.

Hallmarks of a disease, parameters related to said hallmarks (e.g., biomarkers), one or more biological activities associated to said hallmarks etc. are a framework to study a disease or a pathological or medical condition using an integrated/holistic approach.

The hallmarks of an altered physiological state typically include observable changes in various aspects of the body's functioning, which may manifest through symptoms, signs, or laboratory findings.

Altered physiological states typically reflect disruptions in the body's homeostatic mechanisms, leading to deviations from normal physiological parameters. These imbalances may involve alterations in temperature regulation, fluid and electrolyte balance, acid-base balance, glucose metabolism, or other regulatory processes.

Overall, the hallmarks of an altered physiological state provide valuable clues for healthcare providers to identify the underlying cause, assess severity, and guide appropriate interventions to restore normal functioning and promote recovery.

A reference drug is a drug that is commonly selected or chosen as the standard or preferred treatment for a specific medical condition or illness. It is often established based on factors such as its effectiveness, safety profile, cost, and clinical experience. The reference drug serves as a benchmark for comparison with other drugs, especially when assessing generic versions, new treatments, or alternative therapies. It is typically the first drug of choice recommended by medical guidelines or by healthcare providers for treating a particular condition. A reference drug according to the invention is a drug listed in official lists of approved and reference drugs used as comparators in clinical trials and for generic drug approvals issued by regulatory bodies such as FDA, EMA and the like.

In the present description, pathway functional analysis or pathway enrichment and functional analysis (IPA-like analysis) refers to a bioinformatics approach that interprets large-scale omics data (e.g., transcriptomics, proteomics, metabolomics) by: Identifying differentially expressed genes (DEGs) or molecules from experimental data (e.g., RNA-seq, microarrays, mass spectrometry), mapping these genes/proteins/metabolites to known biological pathways (e.g., signalling cascades, metabolic routes); performing enrichment analysis to determine which biological processes or activities, molecular functions, or cellular components are most significantly affected; inferring, if needed, upstream regulators (e.g., transcription factors, cytokines, microRNAs) that may explain observed expression changes; predicting biological effects (e.g., activation or inhibition of pathways, disease associations, drug interactions).

A Z-score is a useful statistical tool for measuring relative position within a dataset, detecting outliers, and standardizing comparisons across different distributions. In the present description the term Z-score (also called a standard score) has the meaning commonly acknowledged in the Art, i.e., it is a statistical measure that describes how far a data point is from the mean of a dataset, expressed in terms of standard deviations. It allows comparison of values from different distributions by standardizing them on a common scale. Z-score is calculated according to the following mathematical formula

$$Z = X - μ / σ$$

Where:
Z=Z-score
X=Individual data point
μ=Mean (average) of the dataset
σ=Standard deviation of the dataset

### DETAILED DESCRIPTION OF THE FIGURES

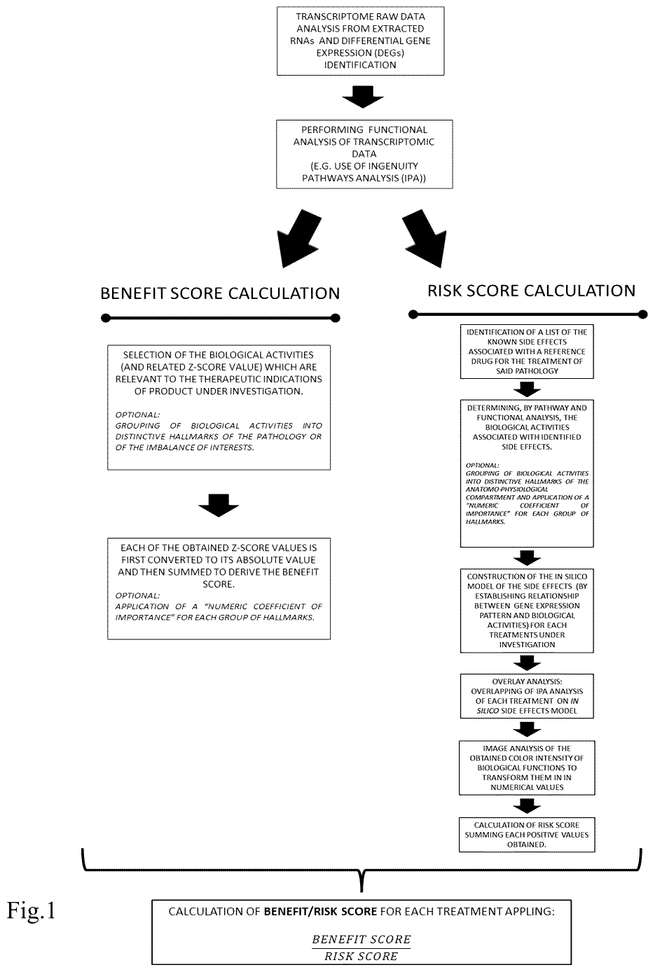

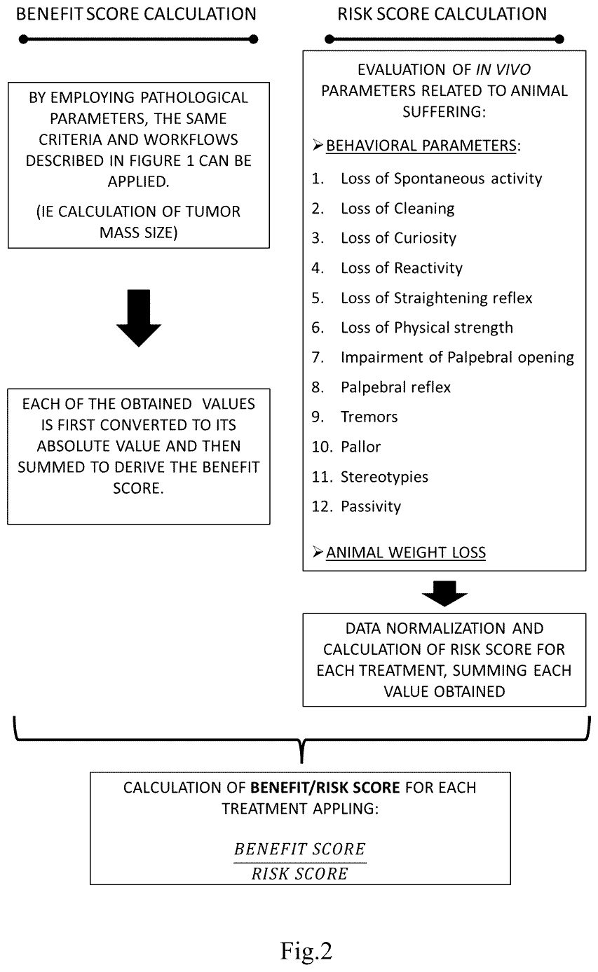

FIG. 1 Flowchart of the procedural process to calculate benefit/risk score on the basis of transcriptional profiles obtained from in vitro or ex vivo experimentation.

FIG. 2 Flowchart of the procedural process to calculate benefit/risk score on the basis of transcriptional profiles obtained from in vivo experimentation.

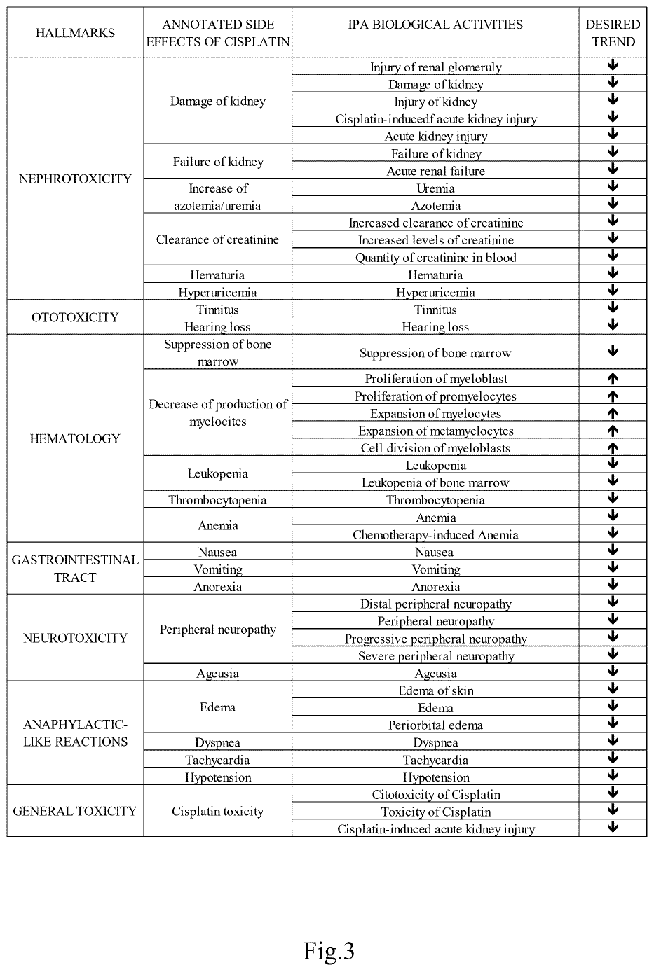

FIG. 3 Annotated side effects of cisplatin and IPA corresponding biological activities. Column “HALLMARKS”: anatomical and functional areas potentially affected by cisplatin toxicity.

Column “ANNOTATED SIDE EFFECTS OF CISPLATIN”: List of cisplatin side effects that have been reported and classified as “common.

Column IPA BIOLOGICAL ACTIVITIES”: Side effects of cisplatin outlined according to the biological activities documented in IPA.

Column “DESIRED TRENDS”: healthy trend of selected biological activities (up-modulation ↑ or down-modulation ↓).

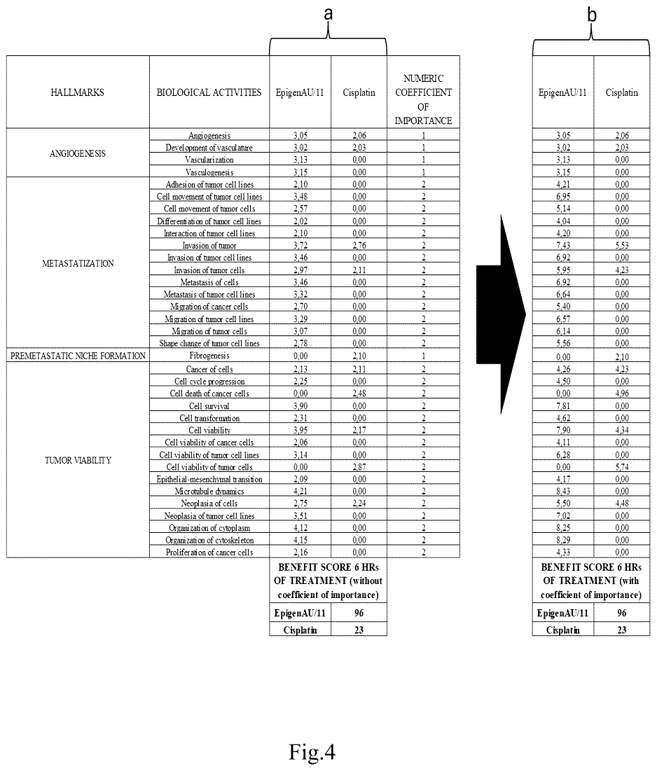

FIG. 4 Calculation of the Benefit score, based on the transcriptional data of EpigenAU/11 and cisplatin, obtained from in vitro and ex vivo studies, without using the optional numeric coefficient of importance (a) and calculation of the Benefit score of EpigenAU/11 and cisplatin by multiplying the obtained Z-scores by their corresponding numeric coefficients of importance (b).

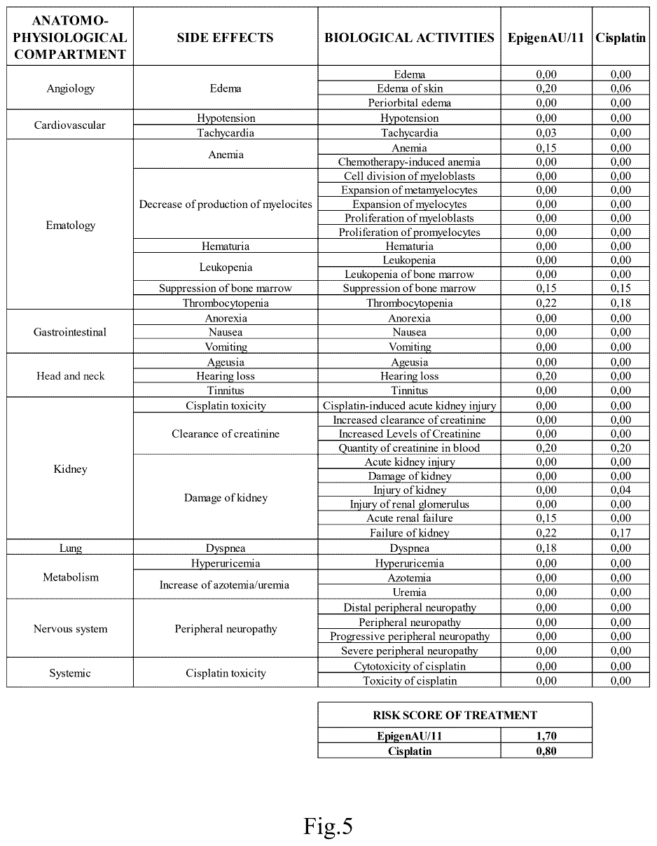

FIG. 5 Calculation of the Risk score of EpigenAU/11 and cisplatin based on the transcriptional data obtained from in ex vivo studies.

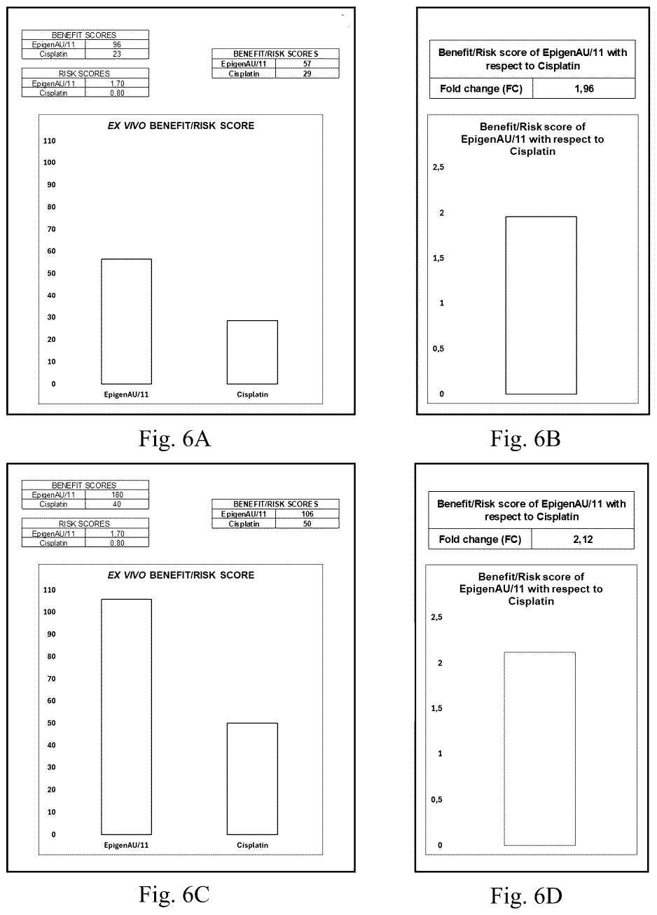

FIG. 6 Benefit/Risk scores obtained with EpigenAU/11 and cisplatin ex vivo treatments after 6 hours, with only mandatory steps according to the description (A). Benefit/Risk scores obtained with EpigenAU/11 and cisplatin treatments after 6 hours with mandatory and optional steps (i.e., multiplying the obtained Z-scores by their corresponding numeric coefficients of importance) according to the description were performed (C). Calculation of the fold change of the score obtained with EpigenAU/11 relative to that obtained with cisplatin: the EpigenAU/11 score is twice that of cisplatin, whether it is calculated using only the mandatory steps (B) or using both the mandatory and optional steps (D).

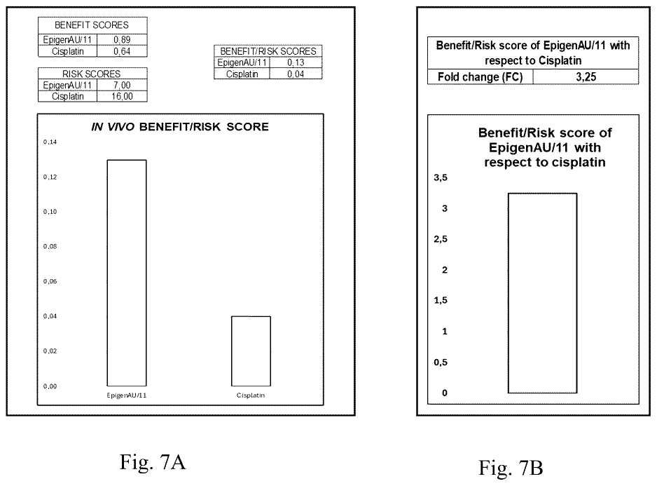

FIG. 7 Benefit/Risk scores obtained by in vivo treatments with EpigenAU/11 and cisplatin on animal model (A). The risk (intended as side effects) was estimated through the observation of behavioural parameters (impacting the behaviour itself, the locomotor system, muscle strength and nervous reflexes) and through the potential weight loss of the animal (see table in example 8.2). The benefit (intended as therapeutic activity) was instead evaluated by taking into consideration the reduction in size of the tumour mass compared to the untreated tumour (see table in example 8.3).

Calculation of the fold change of the score obtained with EpigenAU/11 relative to that obtained with cisplatin: the EpigenAU/11 score is triple that of cisplatin (D).

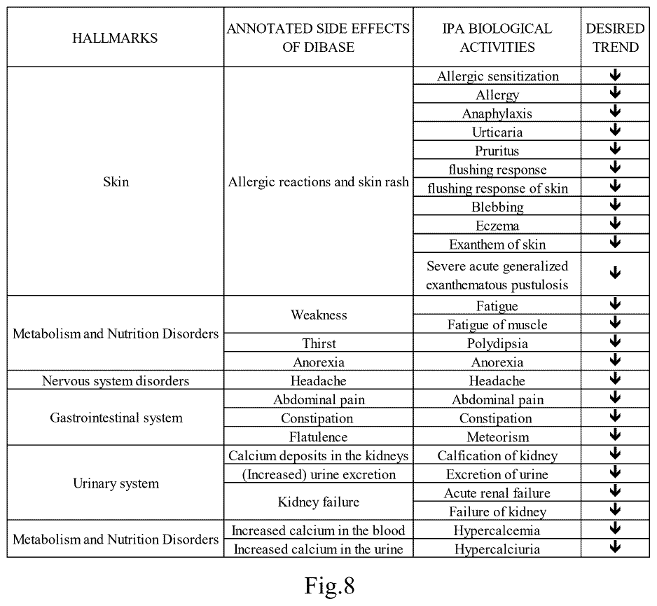

FIG. 8 Annotated side effects of DIBASE and IPA corresponding biological activities. Column “HALLMARKS”: anatomical and functional areas potentially affected by DIBASE toxicity.
Column “ANNOTATED SIDE EFFECTS OF DIBASE”: List of DIBASE side effects that have been reported and classified as “common.
Column IPA BIOLOGICAL ACTIVITIES”: Side effects of DIBASE outlined according to the biological activities documented in IPA.
Column “DESIRED TRENDS”: healthy trend of selected biological activities (up-modulation ↑ or down-modulation ←).

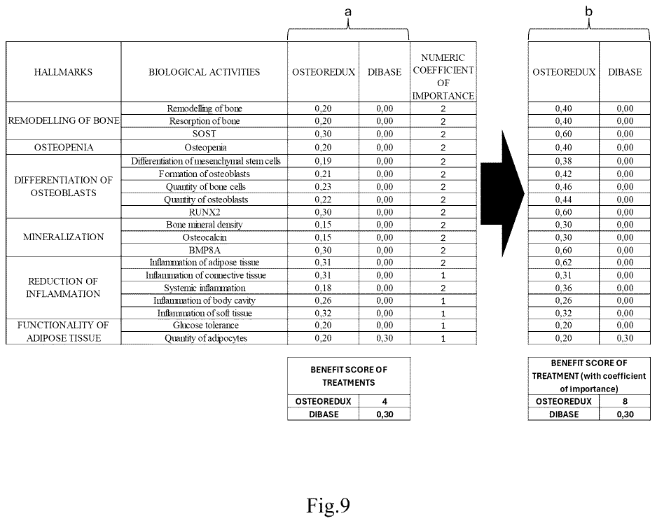

FIG. 9 Calculation of the Benefit score, based on the transcriptional data of Osteoredux and DIBASE, obtained from in vitro and ex vivo studies, without using the optional numeric coefficient of importance (a) and calculation of the Benefit score of Osteoredux and DIBASE by multiplying the obtained Z-scores by their corresponding numeric coefficients of importance (b).

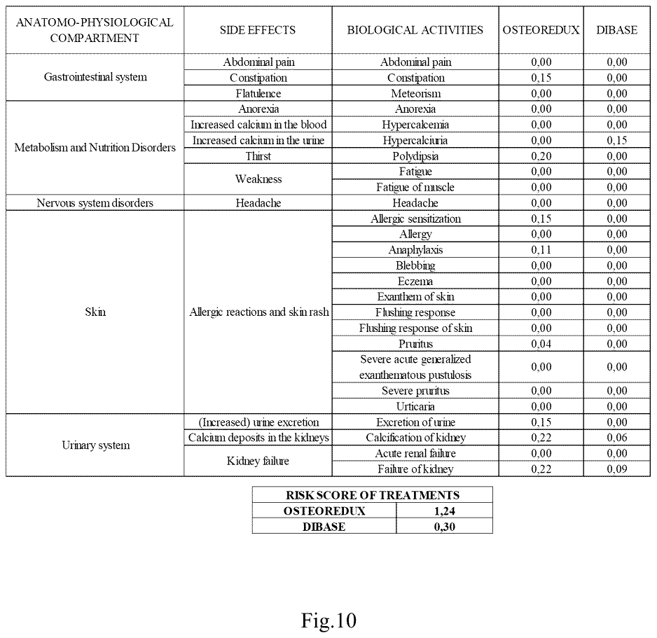

FIG. 10 Calculation of the Risk score of Osteoredux and DIBASE based on the transcriptional data obtained from in vitro studies.

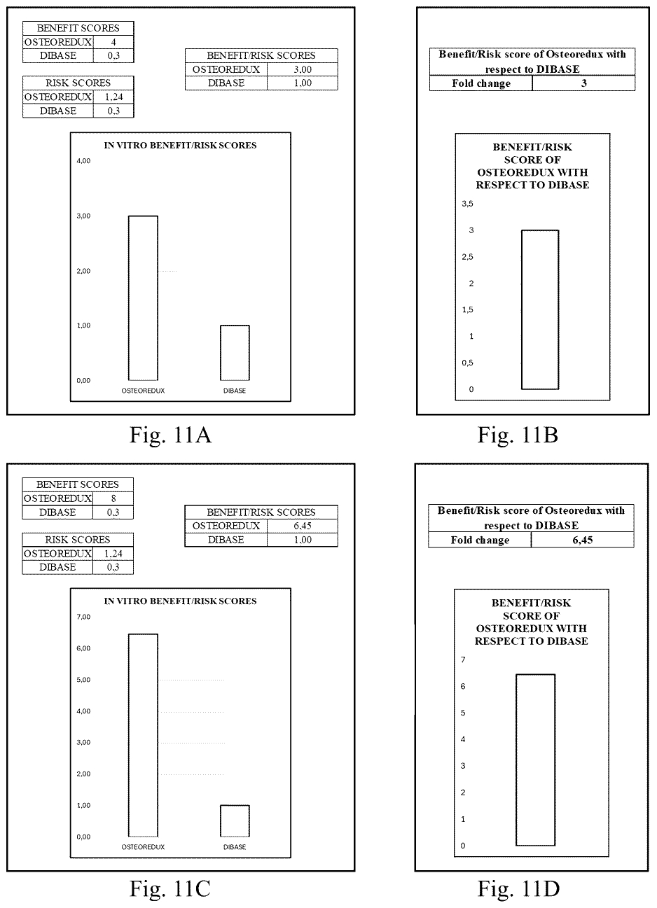

FIG. 11 Benefit/Risk scores obtained with Osteoredux and DIBASE in vitro treatments, with only mandatory steps according to the description (A). Benefit/Risk scores obtained with Osteoredux and DIBASE treatments with mandatory and optional steps (i.e., multiplying the obtained Z-scores by their corresponding numeric coefficients of importance) according to the description were performed (C).
Calculation of the fold change of the score obtained with Osteoredux relative to that obtained with DIBASE: Osteoredux score is three times higher than that of DIBASE when only the mandatory steps are used for the calculation (B), and six times higher than DIBASE when both mandatory and optional steps are included (D).

### DETAILED DESCRIPTION OF THE INVENTION

As stated above, Benefit/Risk evaluation is assessed conceptually, based on qualitative data. The invention, instead, provides a Benefit/Risk Score; i.e., a quantitative and qualitative measure to evaluate the overall value of a medical or beneficial product, intervention, or treatment by comparing its therapeutic or beneficial benefits against its potential risks or harms. It is hence expressed as a ratio between the magnitude of benefits and the magnitude of risks, where benefits refer to the positive therapeutic or beneficial effects of a treatment, such as efficacy in disease management, symptom relief, survival improvement, and quality of life enhancement. Risks, on the other hand, encompass potential harms, including side effects, toxicity, long-term safety concerns, and contraindications.

In the art, to ensure a comprehensive and adaptive evaluation, the 4 Rs approach is often applied: reviewing clinical data to assess benefits and risks, refining methodologies as new evidence emerges, reassessing the benefit-risk balance over time, and reporting findings transparently to stakeholders. This iterative process enhances the accuracy, reliability, and applicability of the Benefit/Risk assessment, supporting evidence-based decision-making in healthcare. The method provided in the present invention, allows for an in vitro or ex vivo prediction of the benefit/risk score of a therapeutic or beneficial product, which is strongly advantageous because it enables early, controlled, and cost-effective evaluation of the product efficacy and safety before advancing to animal models or clinical trials. By providing quantitative and not merely qualitative insights into therapeutic effects and potential toxicity at the cellular level, such method helps refine candidate selection, reduce reliance on in vivo studies, and accelerate regulatory decision-making. This approach can lead to more efficient drug development, minimizing late-stage failures and optimizing patient safety while streamlining the path toward clinical application and approval.

Indeed, the method for the invention is particularly advantageous as it allows a direct comparison between a tested product and a well-established reference drug. By applying the same standardized evaluation criteria to both (product under study and reference drug), this method provides a controlled and quantitative assessment of efficacy and safety, enabling researchers to measure the benefits and risks of the tested product relative to the known profile of the reference drug.

Although the method applies both to synthetic as well as to natural matrices based therapeutic or beneficial products, it is particularly advantageous for the latter. In fact, synthetic drugs' benefits and risks are easier to assess also with the classic methods because they involve single active ingredients with measurable dose-response relationships (known API, known receptor). On the other hand, natural matrices-based therapeutic or beneficial products involve multiple components, synergistic effects, and variability in composition, making it difficult, with classical methods, to easily identify the specific toxicological areas of interest involved.

The present invention provides a new method for calculating the benefit/risk score of a therapeutic or beneficial product, which is also applicable to natural matrices-based products. The method is based on an integrated computer implemented approach utilizing transcriptomic data and advanced biological pathway analysis tools, such as Ingenuity Pathway Analysis (IPA), to identify gene expression changes and modification of relevant biological activities, addressing both therapeutic efficacy and potential adverse effects of said products.

Benefit/risk ratio is a crucial parameter extremely helpful in terms of appreciation of the differences between such key features of a therapeutic option. Nevertheless, such a parameter is currently hard to be reassumed into an objectively calculated single numerical parameter that could facilitate a first approach to the comparison between therapeutic solutions.

The invention provides a method capable of correctly predicting and objectivate the comparison between the benefit/risk ratio of two therapeutic solutions based on exclusively in vitro and ex vivo transcriptomics data. Furthermore, the invention provides an additional method to rationalize and summarize the benefit/risk ratio into a single numerical parameter from data obtained by in vivo on animal models that (see tables in example 8.2 and 8.3). The benefit/risk assessment of a drug in the state of the art involves comparing its observed clinical positive effects (benefits) with its observed clinical negative effects (risks). The process normally begins with clinical trials to evaluate efficacy and safety. Benefits are assessed based on the drug's ability to treat or prevent a condition effectively. Risks are considered by identifying side effects, toxicity, and long-term impacts. Data from preclinical studies, clinical trials, and post-market surveillance help in this evaluation. The drug's safety profile, the severity of side effects, and the severity of the condition being treated are all factored in. Regulatory agencies like the FDA assess the evidence before approval, weighing if the benefits outweigh the risks. Ongoing monitoring ensures continued safety after approval. If risks outweigh benefits, a drug may be recalled or its usage restricted. The invention provides new methods for determining a benefit/risk score of a new potential drug or beneficial product using as product control a known reference drug whose side effects are known.

As stated in the glossary, a reference drug (also called a standard treatment or gold standard therapy) for a disease is a well-established medication that serves as the benchmark for efficacy and safety in treating a specific condition. It is typically the most widely accepted and prescribed treatment for a disease, backed by strong clinical evidence from large-scale trials and real-world data and normally used as the comparator in clinical trials for new drugs. By way of example, regulatory agencies like the FDA and EMA use reference drugs to assess whether a new drug provides added value in terms of efficacy, safety, or tolerability. Therefore, the skilled person can readily identify the reference drug as regulatory bodies maintain official lists of approved and reference drugs used as comparators in clinical trials and for generic drug approvals. By way of example reference drugs are officially listed in FDA's Orange and Purple Books, EMA's EPARs, WHO's Essential Medicines List, and various Health Technology Assessment (HTA) Bodies and clinical guidelines, said lists ensuring consistency, safety, and efficacy in treatment recommendations and drug approvals worldwide.

The methods of the invention can be advantageously used for a predictive evaluation of the benefit/risk ratio of a new product under evaluation, in particular when compared to the benefit/risk ratio obtained with the same methods on a reference drug for the treatment of the same pathology treated by the product under evaluation.

Accordingly, the invention provides discloses a computer implemented method for providing a benefit/risk score of a therapeutical product of interest based on transcriptional in vitro or ex vivo data comprising the following steps
1. performing a transcriptomics analysis by
1.1 providing samples of a biological substrate representing the pathology or pathological state treated by said product and
a. treating one or more of said samples, hereinafter samples a, with said product;
b. treating one or more of said samples, hereinafter samples b, with a reference drug for the treatment of said pathology or pathological state; and
c. using one or more samples, hereinafter samples c, of said biological substrate as relevant control;
1.2 extracting RNA from each of said samples a. b. and c.
1.3 performing a transcriptome raw data analysis from the RNAs extracted in 1.2 and identifying the differentially expressed genes (DEGs), in each of samples a and b, with respect to said samples c, and their expression fold changes with respect to said samples c thereby obtaining fold changes values for each of said DEGs;
1.4 Performing a pathway enrichment and functional analysis of transcriptomic data on the list obtained in 1.3 thereby obtaining numerical values representing the variation in terms of magnitude and directionality (i.e., upregulated or downregulated) of biological activities related to the differential expression of said DEGs in each of samples a and b normalised with respect to samples c;
2. determining the benefit score by
2.1 selecting, among the biological activities of 1.4, the biological activities which are relevant to the therapeutic indications of said product,
2.2 converting the numerical values obtained in 1.4 of said relevant biological activities into absolute values,
2.3 summing said absolute values of each of said biological activities thereby obtaining the benefit score of the product under examination,
3. determining the risk score by
3.1 providing a list of the known side effects associated with a reference drug for the treatment of said pathology,
3.2 determining, by pathway and functional analysis, the biological activities associated with said side effects,
3.3 building a risk in-silico model using pathway and functional analysis by establishing a relationship between gene expression pattern obtained at point 1.3 and the biological activities determined at point 3.2, for samples a and b;
3.4 inputting the relevant fold changes values for each DEGs of samples a. and b. obtained in 1.3 to the risk in-silico model obtained at point 3.3. and using pathway and functional analysis to obtain data representing the direction and the magnitude of the regulation of each biological activity determined at point 3.2 and transforming said data into corresponding numerical values;
3.5 summing each positive value of said numerical values obtained in 3.4 thereby obtaining a final value representing the risk score of the product under examination, wherein, when said final value is <than a predefined positive minimal value, it is automatically corrected to said positive minimal value,
4. providing a benefit/risk score value of the product of interest as the ratio between the benefit score value obtained at point 2.3 and the risk score value obtained at point 3.5

Hence, in the method of the invention, the transcriptional data are generated in vitro or ex vivo, e.g., using suitable biological substrates that are representative models for the pathology or pathological state of interest.

The skilled person will be readily able to select, for generating the necessary transcriptional data according to the method of the invention, suitable biological substrates to be treated with the product of interest or with its relative reference drug, from well known in vitro or ex vivo models. According to a non-limiting example, said in vitro models such as cultured cells, primary cells, human or animal derived cells from relevant tissues (e.g. lung epithelial cells for respiratory pathologies), patient-derived primary cells (e.g. cancer cell lines), immortalised cell lines, stem cell-derived models (e.g. iPSC-derived neurons for neurodegenerative treatments), organ-specific cell lines (e.g. HepG2 for liver metabolism studies), organoid (e.g. gut, brain, kidney etc. organoids mimicking in vivo conditions), spheroids (e.g. 3D cancer or stem cell models), microfluidic “organ on a chip” systems that simulate tissue-level responses and others. Still according to a non-limited example, said ex vivo models can be tissue-based systems such as tissue explants (organotypic slices), human or animal tumour biopsies (e.g., to test anti-cancer products), liver or kidney slice, brain slices, patient derived xenografts (PDX Models). The relevant control c. according to the invention, is selected by the skilled person in order to represent the pathological state without treatment of the product of interest or reference drug.

The method of the invention is applicable in general to therapeutic or beneficial product, the suitable biological sample varying, mutatis mutandis, depending on the therapeutic or beneficial purpose of the product under examination as clear from the present specification and examples. The present specification and examples demonstrate the applicability of the method of the invention to products with extremely different therapeutic purposes, therefore, the method shall not be limited to a specific pathological setup as it can be readily applied by the skilled person to a large number of pathological setups. Indeed, the method of the invention has been developed in order to provide a general procedure protocol that enables developers to assess benefit/risk score to different therapeutic or products and to compare said score to the one of the reference drug of election for said product. In addition, the method herein disclosed, being based on an in vitro or ex vivo system, by providing a fast and reliable of the benefit/risk score, enables developers to select the most promising candidates among new therapeutic or beneficial products under development. The reference drug according to the method is as defined in the glossary, additional studies and evaluations can be made with additional drugs used as reference where desired.

Transcriptomic analysis can be performed according to any conventional technique available to the skilled person suitable for evaluating the whole transcriptome expression profile such as high throughput RNA sequencing, qRT-PCR, microarray and the like. According to the invention, pathway and functional analysis can be performed using commercial or open source tools, one of the most famous tools is QIAGEN Ingenuity® Pathway Analysis (IPA®), other known commercial alternatives to IPA are MetaCore™ (Clarivate Analytics), GeneGo™ (by MetaCore™), QIAGEN OmicSoft®, Isevier® Pathway Studio®, Ingenuity® Variant Analysis (IVA®) (QIAGEN) and open source tools comprise DAVID (Database for Annotation, Visualization, and Integrated Discovery), GSEA (Gene Set Enrichment Analysis), STRING™ (Search Tool for the Retrieval of Interacting Genes/Proteins), KEGG Pathway Analysis, Metascape®, Cytoscape® with enrichment plugins (e.g., ClueGO®, ReactomeFI). Unless otherwise indicated, the present description refers to the last version available at the time of filing of the present invention for each of the above listed tools.

In a preferred embodiment of the invention, when pathway and functional analysis is mentioned in the steps of the method herein disclosed, IPA can be used, in particular, when pathway enrichment and functional analysis of transcriptomic is mentioned (e.g., step 1.4), Core analysis by IPA can be used.

According to the invention, the numerical values in 1.4 indicate the magnitude (e.g., fold changes with respect to control) as well as the directionality (upregulation e.g. by a positive value and downregulation e.g. by a negative value). Said numerical values can be expressed in terms of Z-scores. By way of example, IPA, MetaCore, GSEA, Enrichr provide results in terms of Z-score-based gene regulation analysis. The fold change values obtained in step 1.4 can be expressed as Z-score values to normalize gene expression changes across different datasets or experimental conditions.

When DEGs analysis in 1.4 is made with tools that provide the results in terms of in visual representations, such as color-coded heatmaps, network diagrams, and enrichment plots, as said representations are derived from quantitative data, they can be easily transformed by the skilled person into numerical values using common statistical and computational methods freely available.

When reference is made, e.g. to variation in terms of magnitude and directionality with respect to a control (e.g. samples c.), by way of example, in 1.4, the variation is assessed in terms of folds (magnitude) and up or down regulation (directionality), up regulation will be represented by a positive value, the number of said value will represent the fold change, and down regulation will be represented by a negative value, the number of said value will represent the fold change.

Whole transcriptome expression profile can be made according to any method commonly used by the skilled person, a non-limiting example comprises the use of a Human Clariom™ S Pico Assay HT (Applied Biosystems, ThermoFisher Scientific) on a GeneTitan MC Instrument (Applied Biosystems, ThermoFisher Scientific), following the manufacturer's instructions. CEL Intensity files can be generated by Affymetrix GeneChip Command Console Software (AGCC, ThermoFisher Scientific). Data analyses can be performed using Transcriptomic Analysis Console Software (TAC, ThermoFisher Scientific) that provides quality control analysis, performs normalization and summarization, based on the Signal Space Transformation-Robust Multi-Chip Analysis (SST-RMA) analysis algorithm, and provides a list of differentially expressed genes (Limma Bioconductor package), alternative commercial and/or open source tools known to the skilled person can be used in any part of the generation of the transcriptome expression profile exemplified above. This phase is performed to obtain a list of differentially expressed genes (DEGs), identified based on their expression fold changes with respect to the relevant control experimental condition (i.e., tumour mass without treatments, cell lines not treated with the product of interest and the like).

According to an embodiment of the invention, the analysis of the transcriptional profile can be performed, as stated above, using IPA. The use of IPA allows to readily estimate how and to what extent the modulation of gene expression in a biological system influences the biological activities related to the pathology of interest.

The transcriptional modifications profile obtained is then subjected to a functional pathway enrichment analysis. One of the commercial tools that can be used, and that was used in the examples provided in the present application, is Ingenuity Pathway Analysis (IPA version 94302991, Qiagen) [Krämer et al. (2014)]. When IPA is used, the list of differentially expressed genes and corresponding data measurement values identified in the different experimental conditions is uploaded into the application and the available identifiers are then mapped to their corresponding entity in QIAGEN's Knowledge Base.

By launching “Core Analysis”, significantly perturbed genes, called Network Eligible molecules, are overlaid onto a global molecular network developed from information contained in the QIAGEN Knowledge Base. Networks of Network Eligible Molecules are then algorithmically generated based on their connectivity.

The “core” analysis provides a comprehensive list of approximately top 500 biological activities derived from the generated networks. The associations between biological activities (also indicated herein as biological activities) and genes are always supported by annotations corresponding to scientific peer review publications that substantiate, through the automatic association to a Z-score [Kramer et al. (2014)], which represents the calculated directionality and magnitude of modulation of the biological activities. In essence, this value represents a statistical metric that assesses the similarity between the observed pattern of Differentially Expressed Genes (DEGs) and the expected pattern based on existing literature for a given annotation.

The biological activities that are relevant to the specific pathology under investigation can be selected by interrogating a suitable Al or the selection can be based on the relevant state of the art. The selection of the relevant biological activities is based on the identified known hallmarks of the pathology of interest, applying a Z-score threshold set, accompanied by a statistical significance denoted by a p-value of ≤0.05.

Hallmarks of a given pathology can be readily identified by the skilled person in specialised databases, through pathway and function databases, or through disease specific databases.

The associated Z-score values are then used to indicate the directionality and magnitude of modulation for each biological function (FIGS. 4 and 9).

In case the IPA “core analysis” (or an equivalent analysis thereof using a different open-source or commercial tool) does not yield sufficient relevant information, an alternative available QIAGEN IPA approach called “Overlay analysis” (or an equivalent analysis thereof using a different open-source or commercial tool) can be employed. This analysis focuses on the biological activities identified by the “in-silico model of the patho-physiological state”. The selection of biological activities is structured based on the identified hallmarks of the pathology of interest and can be made by interrogating a suitable Al or can be based on the state of the art.

The “Overlay analysis” establishes relationship between patterns of differentially expressed genes and selected biological activities (always supported by annotations corresponding to scientific peer review publications that substantiate the directionality and magnitude of modulation of the biological activities).

By way of example, this can be done using the following procedure:

Importing the set of biological activities selected from the in-silico model of the patho-physiological state into a new sheet called “my pathway.”

Using the “Build tool” and “Grow tool” to identify Differentially Expressed Genes (DEGs) belonging to the transcriptomic profile under investigation and linked to regulation of the biological activities selected in the previous step.

To determine the expected calculated impact of such experimentally observed modulations of gene expression on biological activities activity, the “Overlay” and “Molecule Activity Predictor” tool (MAP) are employed. The “Prediction” function is activated within MAP tool to calculate the resulting expected modulation of biological activities. (To note, biological activities are defined as “disease and biological functions” in QUIAGEN IPA).

In step 2.1, when present, biological activities that are prima facie in conflict with the acknowledged therapeutic or biological effect of the reference drug selected, are not selected. By way of example, increase of tumorigenesis by an antitumour drug or the like. According to the present description and claims, step 2.2 of converting the numerical values obtained in 1.4 into absolute values, means that, for each value obtained in 1.4, the modulus of said value is considered, i.e., the distance of the number (value) from zero on the number line, regardless of direction. An absolute value is always non-negative.

Summing each value means that a summatory of each value of 2.2 is performed. According to an embodiment of the invention, the method can further comprise: in step 2.1, a step 2.1.a. of clustering said relevant biological activities into hallmarks of the pathology or pathological state treated by the product of interest and providing a numerical coefficient of importance to each of said hallmarks.

In fact, for a more refined the benefit score, in an embodiment of the invention, hallmarks of the pathology of interest can be retrieved, and assigned “numerical coefficients of importance” based on their importance in disease progression and therapeutic impact. Clustering the relevant biological activities selected in 2.1 into said hallmarks and assigning to each hallmark a numerical coefficient of importance, allows a more refined calculation of the relevance of each of said biological activities. Higher coefficients indicate that targeting the hallmark is crucial for effective treatment, while lower coefficients suggest indirect or emerging roles.

Preferably, established hallmarks, i.e., hallmarks officially acknowledged in the art, are used. Hallmarks of a pathology or pathological state can be easily retrieved from the state of the art, alternatively, they can be retrieved by computational methods or interrogating suitable databases, Als (e.g., ChatGPT and others) or dedicated computer programs. By way of example, hallmarks of a pathology can be retrieved using computational methods, by combining bioinformatics tools, machine learning, network analysis, and natural language processing (NLP).

Non limiting examples of coefficients of importance according to the invention are provided below. As a general rule, the hallmarks can be divided into 2 main categories, in brackets, indicative coefficients of importance: High-relevance hallmarks (2): the therapeutic or beneficial product must target said hallmarks for meaningful therapeutic benefit.

Low-relevance hallmarks (1): Useful for complementary effects, but not primary efficacy markers.

Hereinafter non limiting examples of hallmarks of different pathologies together with a coefficient of importance suitable to the method of the invention are provided: Neurodegenerative Diseases (Alzheimer's, Parkinson's, ALS)

| | Relevance | |
|---|---|---|
| | Coefficient | |
| Hallmark | (1/2) | Rationale |
| Amyloid-beta | 2 | Core driver of Alzheimer's disease |
| plaque formation | | progression. |
| Tau hyper- | 2 | Strongly linked to neurodegeneration |
| phosphorylation | | but secondary to amyloid-beta. |
| α-Synuclein | 2 | Main hallmark of Parkinson's, but |
| aggregation | | other mechanisms contribute. |
| Neuroinflammation | 1 | Drives secondary damage but is not |
| (Microglial | | the primary cause. |
| activation) | | |
| Mitochondrial | 1 | Important but often a consequence |
| dysfunction | | rather than a cause. |
| Gut-brain axis | 1 | Emerging but not yet a proven |
| involvement | | therapeutic target. |

### Infectious Diseases (COVID-19, Sepsis, Tuberculosis)

| | Relevance | |
|---|---|---|
| | Coefficient | |
| Hallmark | (1/2) | Rationale |
| Cytokine storm (IL-6, | 2 | Major determinant of severe |
| TNF-α overactivation) | | COVID-19 and sepsis outcomes. |
| ACE2 receptor binding | 2 | Key target for COVID-19 |
| (Viral entry | | antiviral strategies. |
| mechanism) | | |
| Macrophage activation | 1 | Important for immune response |
| | | but not always the key driver. |
| Autophagy regulation | 1 | Plays a role in pathogen clearance |
| in immune response | | but not a direct drug target. |
| Microbiome-immune | 1 | Indirect effects, still an |
| interactions | | emerging field. |

### Oncology (Cancer Types-Breast, Lung, Colon, Leukaemia)

| | Relevance | |
|---|---|---|
| | Coefficient | |
| Hallmark | (1/2) | Rationale |
| Cell cycle dysregulation | 2 | Central driver of uncontrolled |
| (p53, RB1 mutations) | | cell proliferation. |
| PI3K/AKT/mTOR | 2 | Common therapeutic target in |
| pathway activation | | multiple cancers. |
| Immune evasion (PD-L1 | 1 | Key for immunotherapy |
| overexpression) | | response, but variable across |
| | | cancers. |
| Cancer metabolism shifts | 1 | Important but not always a |
| (Warburg effect) | | primary therapeutic target. |

### Cardiovascular Diseases (Atherosclerosis, Heart Failure, Hypertension)

| | Relevance | |
|---|---|---|
| | Coefficient | |
| Hallmark | (1/2) | Rationale |
| LDL cholesterol | 2 | Directly drives atherosclerosis |
| accumulation (Plaque | | and heart disease. |
| formation) | | |
| Renin-Angiotensin- | 2 | Main driver of hypertension and |
| Aldosterone System | | heart failure. |
| (RAAS) activation | | |
| Myocardial fibrosis | 1 | Linked to chronic heart disease but |
| | | secondary to hemodynamic |
| | | changes. |
| Endothelial | 1 | Important contributor but not a |
| dysfunction | | direct drug target. |
| Gut microbiome and | 1 | Emerging research, indirect |
| cardiovascular risk | | relevance. |

### Autoimmune Diseases (Rheumatoid Arthritis, Multiple Sclerosis, Lupus)

| | Relevance | |
|---|---|---|
| | Coefficient | |
| Hallmark | (0-1.0) | Rationale |
| T-cell activation | 2 | Directly regulates autoimmune |
| (Th17, Treg imbalance) | | attack. |
| Autoantibody | 2 | Diagnostic and pathogenic |
| production (ANA, | | markers. |
| RF, anti-CCP) | | |
| Complement system | 1 | Important but secondary in some |
| activation | | autoimmune disorders. |
| Gut microbiota | 1 | Increasingly studied but not yet |
| influence on | | a primary therapeutic target. |
| autoimmunity | | |
| Viral triggers | 1 | Correlated with disease onset but |
| (EBV, CMV infections) | | not a direct treatment target. |

Hallmarks of a given pathology and coefficients of importance thereof can be easily retrieved by the skilled person by suitably interrogating available Als, including ChatGPT. Once hallmarks are retrieved, dedicated programs or statistical or machine learning techniques can be used, and clustering algorithms can be used to group biological activities into hallmarks.

Clustering relevant biological activities into hallmarks of a given pathology can also be readily done by the skilled person by integrating biological data, applying computational methods, and leveraging existing biological knowledge (like pathways or gene ontologies). As stated above, clustering the biological activities into hallmarks of the pathology or pathological state of interest can be done by integrating biological data applying computational methods and leveraging the existing biological knowledge (like pathways or gene ontologies). Numerical coefficients of importance are normally determined based on the biological and/or clinical relevance, or machine learning-based feature importance In addition, the method may further comprise, in step 2.2, multiplying each of the numerical values obtained in 1.4 of said relevant biological activities by the numerical coefficient of importance of the hallmark in which it is clustered provided in 2.1.a before converting said numerical values into absolute values. A numerical coefficient of importance for a hallmark allows for the quantitative assessment of its relevance to a given pathology, enabling a more accurate and objective calculation of a benefit score. This approach helps prioritize therapies or interventions by weighting their effects according to the hallmark's biological significance, ensuring that treatments targeting the most critical disease mechanisms are given greater emphasis. It also facilitates personalized treatment strategies, standardized comparisons across studies, and efficient resource allocation, ultimately enhancing decision-making in research, clinical practice, and drug development.

According to the invention, the side effects considered in 3.1 are the ones indicated as very common (affecting from more than 1 in 10 patients) and common/frequent side effects (affecting from more than 1 in 100 patients) or adverse drug reactions, in the reference's drug leaflet.

To note, the side effects of a drug are reported according to a global pharmacovigilance system and are therefore officially registered, the skilled person can therefore rely simply on the leaflet accompanying the reference drug for the official list of very common and common side effects when carrying out the invention.

In step 3.2, the pathway and functional analysis biological activities associated with said side effects can be determined by inputting in the pathway and functional analysis program said side effects and retrieving the biological activities associated thereto.

In step 3.3, the risk in-silico model using pathway and functional analysis by establishing a relationship between gene expression pattern obtained at point 1.3 and the biological activities determined at point 3.2, for samples a and b can be determined using the tools in the selected pathway enrichment analysis software, by way of example, when IPA is used, the grow tool is suitable for building the risk in-silico model.

In 3.4 the relevant fold changes values can be, as stated above, Z-score values. The indications concerning the Z-score values provided above apply, mutatis mutandis, to step 3.4 of the method.

As for 2.2, also in 3.4, the data are transformed into absolute numerical values.

In the event that the achieved modulation of biological activity signifies no activation of a side effect, the corresponding numerical value is set to zero. Conversely, if the achieved modulation of biological activity indicates the activation of a side effect, the numerical value is converted into its absolute value. In the proposed computer-implemented method for determining a benefit/risk score of a therapeutic or beneficial product, step 3.5 ensures that the risk score has a predefined positive minimal value, even if the computed risk score is initially zero or very low. The reason for this adjustment is that the benefit score must always be higher than the lowest bioactivity value, which serves several important purposes.

The predetermined positive minimal value of 3.5 is introduced in order to prevent mathematically undefined benefit/risk ratio when the risk score is zero (i.e., no detected risk-related bioactivity), the formula would become mathematically undefined due to division by zero. This would prevent meaningful interpretation of the results. By setting a minimal positive risk score, the system avoids division errors while still allowing differentiation between products with different safety profiles.

Additionally, if the risk score were extremely low but not zero, the ratio could be artificially inflated, giving an unrealistic impression of an exceptionally high benefit/risk score. Normalizing the risk score prevents exaggerated values.

In practical applications, comparing multiple drugs or test compounds requires a standardized benefit/risk calculation. If the risk score were to approach zero for some products, their benefit/risk scores would be disproportionately higher than others, even if their actual clinical performance was similar. By enforcing a minimum positive risk value, the method ensures a fair and standardized comparison between different therapeutic candidates.

In addition, the method of the invention takes into account that, in general, no therapeutic or beneficial product is completely risk free, therefore, even if an in vitro or ex vivo transcriptional analysis does not detect strong activation of pathways associated with known side effects, this does not guarantee that the product is completely free of risk in real-world use.

By setting a minimal positive risk value, the system acknowledges inherent uncertainties and ensures that even highly favourable drugs still have a finite, quantifiable risk component.

Finally, the method relies on an in-silico risk model (step 3.3), which predicts risks based on pathway and functional analysis. While this model is powerful, it may not capture all possible adverse effects due to: possible toxicity not detected by transcriptional events, possible limitations in current pathway databases; possible lack of knowledge about all molecular interactions and differences between in vitro/in vivo conditions and actual patient responses.

Enforcing a minimum positive risk score corrects for potential underestimation of risk due to model limitations and helps prevent overconfidence in drug safety.

Therefore, according to the method of the invention said predefined positive minimal value should depend on the scale of measurement and the type of numerical transformation applied to the risk-related biological activities. The chosen value should balance preventing division errors (avoiding infinite or artificially high benefit/risk ratios) while still allowing meaningful differentiation between drugs.

According to an embodiment of the invention said predefined positive minimal value can be a value from 0.001 to 1.0.

By way of example, if the risk score is computed as a sum of positive values (e.g., pathway activation scores, Z-scores, or weighted functional annotations), a reasonable predefined positive minimal value should be small but not negligible.

According to an embodiment, e.g., when the sum of pathway activity scores generally ranges from 0 to 10, said predefined positive minimal value can be 0.1 whereas, if said sum ranges from 1 to 100, said predefined positive minimal value can be 1.0.

As a general rule, the skilled person can select said predefined positive minimal value at 1-10%, e.g., at 5% of the median observed risk score.

When IPA-like pathway analysis is used, said predefined positive minimal value is preferably selected from 0.5 to 1.0.

The predefined positive minimal value can also be selected in relation to the benefit score, e.g., 1-10%, e.g. a 5% of the obtained benefit score.

The skilled person carrying out the method of the invention knows that the benefit score in a quantitative benefit/risk (B/R) assessment represents the total impact of therapeutically relevant biological activities induced by a drug. To ensure that a drug provides a meaningful therapeutic effect, its benefit score must always be higher than the lowest measurable bioactivity value. If this condition is not met, proceeding with the B/R evaluation is not justified. This applies also to the method of the invention.

Additionally, in an embodiment of the invention, the risk in-silico model can also be integrated in order to confer to each side effect associated with the reference drug provided in 3.1, a numerical coefficient of importance, based on the severity of each side effect, based on its clinical relevance, severity, frequency, and impact on patient quality of life.

By way of example, FDA EMA and WHO standards classify side effects by their severity, e.g.

### Classification by Severity (Based on FDA, EMA, and WHO Standards)

Mild→No serious health impact; often resolves without treatment.
Non limiting examples: Nausea, dry mouth, mild headache, drowsiness
Moderate→Interferes with daily activities and may require medical intervention.
Non limiting examples: Dizziness, muscle pain, skin rash, gastrointestinal discomfort
Severe→Serious or life-threatening; requires medical attention.
Non limiting examples: Severe allergic reactions (anaphylaxis), liver failure, heart arrhythmia
Lethal→Causes death or contributes significantly to fatal conditions.
Non limiting examples: Severe drug-induced liver injury (DILI), toxic epidermal necrolysis (TEN)

The numerical coefficient of importance of side effects associated with the reference drug can be, e.g., provided based on the standard clinical grading systems (e.g., CTCAE-Common Terminology Criteria for Adverse Events from the NCI).

By way of examples by assigning severity scores based on grades:
Grade 1 (Mild): Coefficient=1
Grade 2 (Moderate): Coefficient=2
Grade 3 (Severe): Coefficient=3
Grade 4 (Life-threatening): Coefficient=4
Grade 5 (Death): Coefficient=5 or it can be calculated based on the occurrence frequency of each side effects in clinical trials or real-world data, e.g. Common (>10% incidence):

$$Coefficient = 1.$$

Uncommon (1-10% incidence): Coefficient=0.75
Rare (<1% incidence): Coefficient=0.5

By way of example, the risk score adjustment can be readily performed with the aid of a computer software that:
Assigns a numerical coefficient of importance to each side effect based on severity, frequency, quality of life impact, and duration as indicated above,
Calculates the risk contribution using gene expression fold changes,
Computes the total risk score and applies a predefined minimal value,

Generates the benefit/risk score by dividing the benefit score by the risk score.

Displays a summary table with all relevant data.

In other words, the method of the invention may further comprise:
assigning a numerical coefficient of importance to each side effect determined in step 3.1, multiplying the fold change values obtained in step 1.3, which are associated with each side effect, by their corresponding numerical coefficients of importance.

As stated above, said numerical coefficient of importance is calculated, preferably in a computer assisted manner as disclosed above, based on one or more of the following parameters:
severity of the side effect, according to clinical grading systems, frequency of occurrence in clinical data, impact on patient-reported quality of life, duration of the side effect.

According to the invention, step 2.1.a and/or step 3.2 can be carried out using a machine learning model, such as a neural network or support vector machine, trained on known transcriptomic datasets.

It is herein reminded that the value 0 in mathematics and in the present description is not considered as a positive or negative number. Therefore, the correction to the positive minimal value is necessarily applied when a 0 value is obtained in the risk score value provided by the methods herein disclosed.

Preferably the method of the invention is performed also on the reference drug, thereby providing benefit/risk score for the product of interest as well as for the reference drug thereby allowing direct comparison of the benefit/risk scores of the product of interest and its reference drug. Advantageously, the benefit/risk score for the reference drug can be used as a benchmark for evaluating the therapeutic efficacy of the product of interest. Accordingly, the invention also refers to a method for the selection of one or more new therapeutic or beneficial products for clinical development wherein the benefit/risk score for one or more therapeutic or beneficial product of interest for the treatment of a given pathology or pathological state and for the reference drug for the treatment of said pathology or pathological state are provided performing the steps defined above and in the claims, and wherein, when the benefit/risk score value provided for at least one of said therapeutic or beneficial product of interest is equal to or higher than said benefit/risk score value provided for the reference drug, each of said at least one said therapeutic or beneficial product of interest is selected for clinical development.

The invention further discloses a method for providing a benefit/risk score of a therapeutical product of interest for the treatment of a given pathology based on data obtained from in vivo animal models which can be performed in addition to the in vitro or ex vivo method of the invention in order to validate, based on in vivo obtained data, the results obtained with the in vitro or ex vivo method disclosed herein.

Thef further method for providing a benefit/risk score of a therapeutical product of interest for the treatment of a given pathology based on data obtained from in vivo animal models comprise:
I. providing numerical values obtained from the following groups of animal models representing said pathology
a. group treated with said product and
b. group treated with a reference drug for the treatment of said pathology; and
c. relevant control group
said numerical values representing each of the following parameters: the therapeutic efficacy, the behavioural modifications indicative of animal suffering and the body weight-loss indicative of animal suffering observed in each of groups a. and b. normalised with respect to control group c,
II. determining the benefit score by summing said therapeutical efficacy values thereby obtaining the benefit score value of the product under examination,
III. determining the risk score
III.1 identifying as risk parameters the modifications and the weight-loss indicative of animal suffering for which numerical values are provided in 1.
III.2 summing said numerical values of III.1 (with reference to group a.) thereby obtaining a final value representing the risk score of the product under examination, wherein, when said final value is <than a predefined positive minimal value, it is automatically corrected to said positive minimal value
IV. providing a benefit/risk score value of the product of interest as the ratio between the benefit score value obtained at point II and the risk score value obtained at point III.2

The method above can be performed in addition to the in vitro or ex vivo based method of the invention.

Preferably, the method above will be based on pre-existing data obtained from in vivo animal models and the method will use only ethically approved and preferably pre-existing animal study data or data derived from publicly available scientific databases.

The method according to the invention may therefore read as follows:
a computer-implemented method for providing a benefit/risk score of a therapeutic or beneficial product of interest for the treatment of a given pathology based on pre-existing data obtained from in vivo animal models.
I. providing numerical values derived from previously obtained data of in vivo animal models representing said pathology, comprising:
a. a group treated with said product;
b. a group treated with a reference drug for the treatment of said pathology; and
c. a relevant control group; Wherein said numerical values represent:
i. therapeutic efficacy;
ii. behavioural modifications indicative of treatment effects; and
iii. body weight changes observed in groups a and b, normalized with respect to control group c,
II. determining the benefit score by summing said therapeutic efficacy values, thereby obtaining the benefit score of the product under examination.
III. determining the risk score:
III.1. identifying risk parameters using said numerical values related to behavioural modifications and body weight changes and
III.2. summing said numerical values related to adverse effects (as identified in III.1) for group a, thereby obtaining a final value representing the risk score of the product under examination. If the final value is below a predefined positive minimal threshold, it is adjusted to said minimal value.
IV. providing a benefit/risk score by calculating the ratio between the benefit score (obtained in II) and the risk score (obtained in III.2).

When animal data are used, only data obtained from animals treated according to ethically approved protocols will be used.

As stated above, advantageously, the method based on data obtained from in vivo models, can be combined with the method based on in vitro or ex vivo data in order to further validate the predictive results obtained therefrom.

The data obtained from an in vivo model in the examples below, that validate the results obtained in vitro or ex vivo with the method of the invention, demonstrate that the in vitro or ex vivo method of the invention does, indeed, provide reliable benefit/risk scores thereby rendering unnecessary in vivo experiments.

Also the method disclosed herein for the selection of one or more new therapeutic or beneficial products for clinical development can further comprise the calculation of the benefit/risk score for each tested product and reference drug with the method based on data obtained from in vivo models disclosed above, when the benefit/risk score value provided for at least one of said therapeutic or beneficial product of interest with both methods (in vitro or ex vivo as well as based on data obtained from in vivo models) is equal to or higher than both the relative benefit/risk score values (in vitro or ex vivo as well and based on data obtained from in vivo models) provided for the reference drug, each of said at least one said therapeutic or beneficial product of interest is selected for clinical development.

Equal to or higher, herein means that the value is numerically equal higher, i.e., the benefit/risk score value of the tested product is numerically equal or higher than the benefit/risk score of the reference drug, preferably, it is even of statistically significant superiority.

All the additional steps disclosed for the in vitro or ex vivo based method above, apply, mutatis mutandis to the method based on data obtained from in vivo models as disclosed herein. Therapeutic efficacy parameters depend on the pathology of interest, e.g., for an anticancer drug said parameters are mainly represented by the tumour mass reduction and, optionally, by the presence or absence of metastasis; for an antidepressant drug said parameters can comprise behavioural modifications indicative of an amelioration of the depression gravity and so on. The parameters hence will be parameters commonly correlated with the assessment of therapeutic efficacy in the treatment of a given pathology. Parameters indicative of behavioural modifications indicative of animal sufferings are parameters that are codified by standard tests commonly used in the art such as, e.g., Irwin test, used in official protocols for evaluating animal suffering in pre-clinical trials or housing. Even in this case the benefit/risk score can be provided also for the reference drug. As stated above, preferably, when applicable, both methods are carried out so to validate the results obtained with either one of them.

In preferred embodiments of the invention, in the methods as herein described said therapeutic or beneficial product of interest is a product comprising one or more natural matrices, such as natural matrices obtained from eukariotic, prokariotic sources such as plant, marine, bacterial, fungi, yeasts, animal, natural sources; by way of example, one or more of: cut or pulverized plant parts, plant extracts, fractions of said extracts, microorganisms, honey, propolis, silk, wax, plant resins, plant gums, plant exudates, vegetable oils, vegetable essential oils, animal tissues lysates, or plant or animal fluids. In any part of the present description and claims the term comprising can be substituted by the term consisting of.

In any part of the description or in the claims, when calculations are made, this can be carried out in a computer implemented mode.

In any part of the description, when an internet address or URL is provided, it relates to the information retrieved from said internet address available at the filing date of the present application, i.e., at the latest version of the content at said internet address at the filing date of the present application.

In any part of the description or in the claims, when reference is made to commercial products, the symbols TM (™) or © are considered as implicit and can be added to each of said products at any time.

In any part of the description or in the claims, when reference is made to “a computer implemented method for providing a benefit/risk score” the sentence can be substituted by “a computer-implemented method for producing a therapeutic or beneficial product, comprising determining its benefit/risk score”.

Examples are reported below which have the purpose of better illustrating the embodiments disclosed in the present description, such examples are in no way to be considered as a limitation of the previous description and the subsequent claims, further, the examples below report all the studies performed on the product of the invention and support all the subject-matter claimed.

### EXAMPLES

### 1. Composition of the Tested Products

Product A, herein, also Osteoredux, for the treatment of bone fragility
Coral skeleton powder 32% w/w
Avian Egg shell powder 30.2% w/w
Dried Coral skeleton plus lemon juice (comprising calcium citrate) powder 13% w/w
Agaricus bisporus powder 4.65% w/w
Equisetum arvense flowering tops dry extract 2% w/w
Malpighia punicifolia carried by inulin dry extract 2.0% w/w
Cetraria islandica powder 2% w/w
Agave sisalana leaves powder 12% w/w
Acacia senegal powder 2.15% w/w
Product B, herein, also EpigenAU/11 for the treatment of cancer.
36.05% in weight of freeze-dried component 1
63.06% in weight of freeze-dried component 2
0.89% in weight of freeze-dried component 3.

### Component 1

Laurus nobilis leaves 25% w/w
Withania somnifera roots 25% w/w
Filipendula vulgaris leaves and flowers 25% w/w
Brassica oleracea L. botrytis cymosa seeds 25% w/w
Coextracted in water

### Component 2

Cynara scolymus L. leaves 14.30% w/w
Curcuma longa L. roots 42.85% w/w
Tanacetum parthenium L. flowers 42.85% w/w
Coextracted in water

### Component 3

Agave sisalana leaves freeze-dried extract.

### 2. Summary of Protocols Used

| | PRODUCT A | PRODUCT B |
|---|---|---|
| GENE EXPRESSION | Gene expression analysis | Gene expression analysis |
| ANALYSIS | At the end of described treatment periods, | At the end of described treatment periods, |
| | cells were washed with 100 μl PBS and | tumour fragments were homogenized using Lysing |
| | lysed and collected in RLT buffer (Qiagen, | Matrix Tubes J (MP Biomedicals) in RLT buffer |
| | 1053393) added with β-mercaptoethanol | (Qiagen, 1053393) added with β-mercaptoethanol |
| | (Sigma, M3148) and DX reagent (Qiagen, | (Sigma, M3148) and DX reagent (Qiagen, 19088) |
| | 19088) for gene expression analysis | for gene expression analysis experiments. Total |
| | experiments. Total RNA was extracted from | RNA was extracted from cells lysates using an |
| | cells lysates using an QIAsymphony RNA Kit | QIAsymphony RNA Kit (Qiagen,) with the |
| | (Qiagen,) with the QIAsymphony SP | QIAsymphony SP instrument (Qiagen). |
| | instrument (Qiagen). | The quality and quantity of RNA was |
| | The quality and quantity of RNA was | determined by A230, A260, A280 and A320 |
| | determined by A230, A260, A280 and A320 | measurements on Varioskan ™ LUX |
| | measurements on Varioskan ™ LUX | multimode microplate reader (Thermo |
| | multimode microplate reader (Thermo | Scientific ™). Integrity of RNA was checked |
| | Scientific ™). Integrity of RNA was checked | using a 2100 expert_Eukaryote Total RNA |
| | using a 2100 expert_Eukaryote Total RNA | Nano Kit (Agilent). Whole transcriptome |
| | Nano Kit (Agilent). Whole transcriptome | expression profile was evaluated using a |
| | expression profile was evaluated using a | Human Clariom ™ S Pico Assay HT (Applied |
| | Human Clariom ™ S Pico Assay HT (Applied | Biosystems, ThermoFisher Scientific) on |
| | Biosystems, ThermoFisher Scientific) on a | a GeneTitan MC Instrument (Applied Biosystems, |
| | GeneTitan MC Instrument (Applied Biosystems, | ThermoFisher Scientific), following the |
| | ThermoFisher Scientific), following the | manufacturer's instructions. Briefly, 6 ng |
| | manufacturer's instructions. Briefly, 6 ng | of total RNA was used to generate cDNA, |
| | of total RNA was used to generate cDNA, | then fragmented and labelled cDNA was |
| | then fragmented and labelled cDNA was | hybridized to a Human Clariom S 96-array |
| | hybridized to a Human Clariom S 96-array | plate for 17 h at 45° C. Arrays were washed, |
| | plate for 17 h at 45° C. Arrays were washed, | stained and then scanned using the GeneTitan |
| | stained and then scanned using the GeneTitan | MC Instrument (Applied Biosystems, |
| | MC Instrument (Applied Biosystems, | ThermoFisher Scientific) and CEL Intensity |
| | ThermoFisher Scientific) and CEL Intensity | files were generated by Affymetrix |
| | files were generated by Affymetrix | GeneChip Command Console Software (AGCC, |
| | GeneChip Command Console Software (AGCC, | ThermoFisher Scientific). |
| | ThermoFisher Scientific). | |
| TRANSCRIPTOMICS | Transcriptomics data analysis | Transcriptomics data analysis |
| DATA ANALYSIS | Data analysis was performed using | Data analysis was performed using |
| | Transcriptomic Analysis Console Software | Transcriptomnic Analysis Console Software |
| | (TAC, ThermoFisher Scientific) that | (TAC, ThermoFisher Scientific) that |
| | provides quality control analysis, performs | provides quality control analysis, performs |
| | normalization and summarization, based | normalization and summarization, based |
| | on the Signal Space Transformation- | on the Signal Space Transformation- |
| | Robust Multi-Chip Analysis (SST-RMA) | Robust Multi-Chip Analysis (SST-RMA) |
| | analysis algorithm, and provides a list of | analysis algorithm, and provides a list of |
| | differentially expressed genes (Limma | differentially expressed genes (Limma |
| | Bioconductor package, p-value ≤ 0.05). | Bioconductor package, p-value ≤ 0.05). |
| CELL | Human, adipocyte-derived, mesenchymal | Biopsies obtained from tumour masses generated |
| BASED ASSAY | stem cell lines (hADMSC), capable of | by subcutaneous inoculation in nude mice of FaDu |
| | differentiating into osteoblasts and | cells, a squamous cell carcinoma line (obtained |
| | mineralize the extracellular matrix | from the American Type Culture Collection-ATCC |
| | (ECM) were used in this study. These cells | HTB-43), capable of tumorigenesis The biopsies |
| | were obtained during general surgery from | were immediately frozen in liquid nitrogen after |
| | three different patients (PA42, PA59 and | explant, and then transferred to −80° C. Upon |
| | PA69) (Romagnoli et al, “In vitro Behaviour | thawing in Eagle's Minimum Essential Medium |
| | of Human Adipose Tissue-Derived Stem | (EMEM) (ATCC, 30-2003), they were sectioned into |
| | Cells on Poly(ε-caprolactone) Film for Bone | approximately 40 mg pieces and cultured for either |
| | Tissue Engineering Applications”, BioMed | 6 hours (for gene expression analysis) or 72 hours |
| | Research International, vol. 2015, Article ID | (for cytofluorimetric analysis) in a humidified |
| | 323571, 12 pages, | incubator at 37° C., with an atmosphere enriched |
| | 2015. doi.org/10.1155/2015/323571). | with 5% CO2, in the absence or presence of |
| | These cell lines have been characterized | PRODUCT D at 50 mg/ml or Cisplatin at 666 μM. |
| | with respect to the main stemness markers | For gene expression analysis, at the end of the 6- |
| | of mesenchymal stem cells (CD44, CD105, | hour treatment period, tumour fragments were |
| | and STRO1) and by studying their | homogenized using Lysing Matrix Tubes J in RLT |
| | multipotency toward osteogenic | buffer and followed the previously described |
| | phenotypes at the Department of Surgery | process. For cytofluorimetric analysis, at the end of |
| | and Translational medicine of the | the 72-hour treatment period, the masses were |
| | University of Florence. | collected and fragmented using a scalpel to create |
| | hADMSCs were cultured in a growth | smaller pieces. These fragments underwent further |
| | medium (GM) and grown to 70-80% | processing to obtain single-cell suspensions using |
| | confluence. Afterwards, the cells were | the gentleMACS Octo Dissociator with Heaters |
| | seeded in 24-well plate at a concentration | (Miltenyi Biotec, 130-096-427) in combination with |
| | of 1 × 10⁵ cells/well. After a week, the GM | a tissue dissociation kit (Miltenyi Biotec, 130-095- |
| | was replaced with osteogenic medium | 0929), following the manufacturer's instructions. |
| | (OM) containing the fluorophore calcein | Cellular debris was removed using the MACS |
| | 1 μg/mL and incubated for 28 days with or | Miltenyi Biotec Debris Removal Solution (Miltenyi |
| | without product C. The medium with or | Biotec, 130-109-398), and red blood cells were |
| | without the product C was refreshed twice | eliminated using Red Blood Cell Lysis Solution (10×) |
| | a week. | (Miltenyi Biotec, 130-095-0929) according to the |
| | | manufacturer's protocol. |
| | | Following dissociation, cells were resuspended and |
| | | stained with CD45 Monoclonal Antibody (30-F11), |
| | | PE-Cyanine7, eBioscience ™ (Invitrogen, 25-0451- |
| | | 82) at a concentration of 0.125 μg/test for 30 |
| | | minutes on ice with occasional shaking. Cells were |
| | | then washed in cold DPBS (ThermoFisher, 14190- |
| | | 094) at 1200 rpm for 5 minutes. CD45 Monoclonal |
| | | Antibody was utilized to exclude all hematopoietic |
| | | cells except mature erythrocytes and platelets. |
| | | After removing the supernatant, cells were stained |
| | | with Alexa Fluor ® 488 Annexin V/Dead Cell |
| | | Apoptosis Kit (ThermoFisher, V13241, A13201). |
| | | Subsequently, the cells were resuspended in 500 μl |
| | | 1X annexin-binding buffer and analysed using an |
| | | Attune NxT flow cytometer (Life Technologies). |
| DEFINITION OF THE | The alteration of healthy physiological state | The alteration of healthy physiological state |
| PATHOPHYSIOLOGICAL | features of state-of-the-art “Osteoporosis” | features of state-of-the-art “Cancer” were |
| HALLMARKS | were considered with particular attention | considered with particular attention to the |
| OF THE DISEASE | to the following areas involved: | following areas involved: |
| WITH WHICH TO | Remodelling of bone | Cell damage |
| INTERROGATE IPA | Osteopenia | Energetic metabolism and insulin sensitivity |
| | Differentiation of osteoblasts | Epithelial-mesenchymal transition |
| | Mineralization | Growth factor |
| | Reduction of inflammation | Inflammation |
| | Reduction of adipose tissue | Regulation of mitosis/proliferation |
| | This knowledge was used to interrogate | Stemness |
| | IPA via the “IPA Bioprofiler” tool, using the | This knowledge was used to select and classify IPA |
| | following keywords: osteoporosis, | core analysis outputs (i.e., Canonical pathways). |
| | postmenopausal osteoporosis, calcification | |
| | of bone, osteoblast and osteoclast | |
| | differentiation, bone mineral density. | |

### 3. Techniques and Settings for Calculating Benefit/Risk Score of Products A and B.

### 3.1 In Vitro or Ex Vivo Sample Treatments

### Bone Fragility: PRODUCT A-Osteoredux

Human, adipocyte-derived, mesenchymal stem cell lines (hADMSC), capable of differentiating into osteoblasts and mineralize the extracellular matrix (ECM) were used in this study. These cells were obtained during general surgery from three different informed patients (PA42, PA59 and PA69) (Romagnoli et al, “In vitro Behaviour of Human Adipose Tissue-Derived Stem Cells on Poly(ε-caprolactone) Film for Bone Tissue Engineering Applications”, BioMed Research International, vol. 2015, Article ID 323571, 12 pages, 2015. doi.org/10.1155/2015/323571). These cell lines have been characterized with respect to the main stemness markers of mesenchymal stem cells (CD44, CD105, and STRO1) and by studying their multipotency toward osteogenic phenotypes at the Department of Surgery and Translational medicine of the University of Florence.

hADMSCs were cultured in a growth medium (GM) and grown to 70-80% confluence. Afterwards, the cells were seeded in 24-well plate at a concentration of 1×105 cells/well. After a week, the GM was replaced with osteogenic medium (OM) containing the fluorophore calcein 1 μg/mL and incubated for 28 days with or without Osteoredux or reference drug DiBase. The medium with or without the product C was refreshed twice a week.

### Cancer Area: PRODUCT B-EpigenAU/11

To perform the experiments, ex vivo tumour masses were generated from the FaDu head and neck squamous carcinoma cell line implanted in immunocompromised mice. Once the tumours reached an adequate size, they were excised, divided into 40 mg portions, and treated in triplicate with EpigenAU/11 or reference drug Cisplatin for 6 hours. Subsequently, the masses were lysed, and RNA was extracted for transcriptional analysis.

### 3.2 Transcriptome Raw Data Analysis

Whole transcriptome expression profile was evaluated. In particular, a Human Clariom™ S Pico Assay HT (Applied Biosystems, ThermoFisher Scientific) on a GeneTitan MC Instrument (Applied Biosystems, ThermoFisher Scientific), following the manufacturer's instructions were used. CEL Intensity files were generated by Affymetrix GeneChip Command Console Software (AGCC, ThermoFisher Scientific). Data analyses were performed using Transcriptomic Analysis Console Software (TAC, ThermoFisher Scientific) that provides quality control analysis, performs normalization and summarization, based on the Signal Space Transformation-Robust Multi-Chip Analysis (SST-RMA) analysis algorithm, and provides a list of differentially expressed genes (Limma Bioconductor package). This phase allows to obtain a list of differentially expressed genes (DEGs), identified based on their expression fold changes with respect to the relevant control experimental condition (i.e., tumour mass without treatments, cell lines not treated with the product of interest and the like). Detailed protocols are in the table above.

### 3.3 Ingenuity Pathway Analysis IPA (Quiagen IPA) Analysis of Transcriptional Profile

The use of IPA allows to estimate how and to what extent the modulation of gene expression in a biological system influences the biological activities related to the pathology of interest. The transcriptional modifications profile obtained were subjected to a functional pathway enrichment analysis. Ingenuity Pathway Analysis (IPA version 94302991, Qiagen) [Krämer et al. (2014)] was used. For each benefit/risk assessment (e.g., product A, product B) the list of Differentially expressed genes and corresponding data measurement values identified in the different experimental conditions were uploaded into the application.

Available identifiers were mapped to their corresponding entity in QIAGEN's Knowledge Base.

By launching IPA “Core Analysis”, significantly perturbed genes, called Network Eligible molecules, were overlaid onto a global molecular network developed from information contained in the QIAGEN Knowledge Base. Networks of Network Eligible Molecules were then algorithmically generated based on their connectivity.

The core analysis provides a comprehensive list of approximately top 500 biological activities derived from the generated networks. The IPA associations Biological activities-genes are always supported by annotations corresponding to scientific peer review publications that substantiate, through the automatic association of a Z-score [Krämer et al. (2014)], the calculated directionality and magnitude of modulation of the biological activities (in the invention reference is made to biological activities, IPA denomination for the same is “biological activities”). In essence, the value obtained represents a statistical metric that assesses the similarity between the observed pattern of Differentially Expressed Genes (DEGs) and the expected pattern based on existing literature for a given annotation.

The biological activities that are relevant to the specific pathology under investigation were selected based on the relevant state of the art but the same can be done by interrogating suitable Als. The selection of biological activities was based on the identified known hallmarks of the pathology of interest, applying a Z-score threshold set, accompanied by a statistical significance denoted by a p-value of ≤0.05.

The associated Z-score values was then used to indicate the directionality and magnitude of modulation for each biological function (FIGS. 4 and 9).

In case the IPA core analysis did not yield sufficient relevant information, an alternative approach called “Overlay analysis” was employed. This analysis focused on the biological activities identified by the “in-silico model of the patho-physiological state”. The selection of biological activities was structured based on the identified hallmarks of the pathology of interest. This can be made by interrogating a suitable Al or can be based on the state of the art.

The “Overlay analysis” is structured by establishing relationship between patterns of differentially expressed genes and selected biological activities (always supported by annotations corresponding to scientific peer review publications that substantiate the directionality and magnitude of modulation of the biological activities).

This was be done using the following procedure:

The set of biological activities (called functions in IPA), selected from the in-silico model of the patho-physiological state were imported into a new sheet called “my pathway.”

The “Build tool” and “Grow tool” to identify Differentially Expressed Genes (DEGs) belonging to the transcriptomic profile under investigation and are linked to regulation of the biological activities selected in the previous step was used.

To determine the expected calculated impact of such experimentally observed modulations of gene expression on biological activities activity, the “Overlay” and “Molecule Activity Predictor” tool (MAP) were employed. The “Prediction” function was activated within MAP tool to calculate the resulting expected modulation of biological activities.

### 3.4 Image Analysis of the Obtained Color Intensity of Biological Activities

As the “Overlay analysis” does not directly calculate the Z-score for each biological activity (function in IPA). Therefore, the intensity of the modulation signal was translated into a numerical value. This was achieved by converting the use of dedicated app: “IPAmap_Parser”.

The app is a web port app of Pipeline Pilot designed to assign a score, called a z-score, to genes and biofunctions based on their colouring within a biological pathway generated by QIAGEN's Ingenuity Pathway Analysis software. The key step of the algorithm is the conversion from the RGB colour model to the LAB [xrite.com/it-it/blog/lab-color-space] model, a colorimetric encoding that allows for the recording of colour intensity, not just the RGB composition. This conversion takes place within a Pipeline Pilot “component” that utilizes a procedure written in R software, relying on specific functionalities of the colorspace package
[cran.r-project.org/web/packages/colorspace/index].

### 4. Risk Score Calculation

### 4.1 Definition of Side Effects

Officially recorded very common or common (≥1 out of 10 patients or >1 out of 100 patients) side effects were identified by consulting specific sources:
torrinomedica.it
drugs.com/
specific leaflet of the reference product

### Specific Sources for Area of Interest

### Osteoarthritis:

torrinomedica.it/schede-farmaci/kenacort/
drugs.com/sfx/triamcinolone-side-effects.html

### Bone Fragility:

torrinomedica.it/schede-farmaci/dibase/-drugs.com/sfx/cholecalciferol-side-effects.html

### Cancer Area:

torrinomedica.it/schede-farmaci/cisplatino-3/
drugs.com/sfx/cisplatin-side-effects.html
   4.2 Identification of IPA Biofunctions Browsing them from the Terms Used to Annotate the Side Effects

The information found in the aforementioned resources is used to identify BFs (i.e., biological activities) related to the identified side effects and to interrogate IPA through the following procedure:
side effects terms were inputted, one by one, in the “disease and functions” query box and the search was then launched.

The analysis of the obtained resuming table was filtered for identifying disease/function that come from many lines of evidence. The source for the relationship was the Ingenuity Knowledge Base, including curation from journal articles, OMIM, JAX and ClinicalTrials.gov. In-silico model was limited to genes and mRNAs. The tool was therefore able to associate with each biological activity a defined number of genes whose modulation is able to influence the modulation of the biological activity itself (in this specific case, able to represent the side effect).

### 4.3 Overlay Analysis

Overlay analysis focuses on the biological activities identified by the “in-silico model of side effects”. The selection of biological activities is structured based on the identified side effects of reference treatment.

The “Overlay analysis” is structured by establishing relationship between patterns of differentially expressed genes and selected biological activities (always supported by annotations corresponding to scientific peer review publications that substantiate the directionality and magnitude of modulation of the biological activities) using the following procedure:

The set of biological activities selected from the in-silico model of the SIDE EFFECT PROFILE was imported into a new sheet called “my pathway.”

The “Build tool” and “Grow tool” were used to identify Differentially Expressed Genes (DEGs) belonging to the transcriptomic profile under investigation and are linked to regulation of the biological activities selected in the previous step.

To determine the expected calculated impact of such experimentally observed modulations of gene expression on biological activities activity, the “Overlay” and “Molecule Activity Predictor” tool (MAP) are employed. The “Prediction” function is activated within MAP tool to calculate the resulting expected modulation of biological activities.

### 4.4 Image Analysis of the Obtained Color Intensity of Biological Activities

As stated above, as the “Overlay analysis” does not directly calculate the Z-score for each biological function, the intensity and directionality of the modulation was translated from a colour heatmap into a numerical value using the dedicated app: “IPAmap_Parser” so to obtain a Z-score representing the intensity and the directionality of each modulation.

### 4.5 Calculation of Global Risk Score

Risk score was then calculated by summing each positive Z-score value obtained from overlay analysis (to indicate that a given side effect was induced by the treatment).

### 5. Benefit Score Calculation

### 5.1 Core Analysis, Biological Activities Selection and Export

Using core analysis, the suitable biological activities (biofunctions to use IPA language) were selected taking into account those whose trend is consistent with the therapeutic indications of the product under investigation. The relevant biological activities for a specific therapy can be identified through Pathway Enrichment Analysis, using a suitable Al for literature mining, or using pathology specific databases.

The following biological activities were selected for each pathological area examined and used with IPA

### Cancer Area

The well-known alteration of healthy physiological state features associated with Cancer were considered with particular attention to the following areas involved:
Cell damage
Energetic metabolism and insulin sensitivity
Epithelial-mesenchymal transition
Growth factor
Inflammation
Regulation of mitosis/proliferation
Stemness
The above was used to select and classify IPA core analysis outputs

### Bone Fragility

The well-known alteration of healthy physiological state features of “Bone fragility” were considered with particular attention to the following areas involved:
Remodelling of bone
Osteoporosis
Differentiation of osteoblasts
Mineralization
Reduction of inflammation
Reduction of bone adipose tissue

The above was used to interrogate IPA via the “IPA Bioprofiler” tool, using the following keywords: osteoporosis, postmenopausal osteoporosis, calcification of bone, osteoblast and osteoclast differentiation, bone mineral density.

BFs were grouped in activity hallmarks (and, optionally, each hallmark is given a weight coefficient based on its importance in the pathogenesis of interest). Hence BFs Z-score values are transformed in absolute values and the lists thus obtained are exported in a table which reports the specification of the biofunctions and their relative modulation values. The sum of each value for each treatment are considered as the benefit score.

### 6. Benefit/Risk Score Calculation

Once the risk scores and the benefit scores were calculated, they were applied to the following formula:

(BENEFIT SCORE)/(RISK SCORE)

The value of the ratio must therefore be interpreted as: the higher the value obtained, the greater the safety of the administered treatment.

### 7. New Benefit/Risk Assessment Product A (Computer Implemented/Assisted)

### 7.1 Data Assembly

### 7.1.1 Ex Vivo Sample Treatment and RNA Extraction (Steps 1.1 and 1.2)

To perform the experiments, ex vivo tumour masses were generated from the FaDu head and neck squamous carcinoma cell line implanted in immunocompromised mice. Once the tumours reached an adequate size, they were excised, divided into 40 mg portions, and treated in triplicate with EpigenAU/11 (samples a.) or cisplatin (samples b.) or untreated (samples c.) for 6 hours. Subsequently, the masses were lysed, and RNA was extracted according to standard protocols for transcriptional analysis.

### 7.1.2 Transcriptome Raw Data Analysis (Step 1.3)

Whole transcriptome expression profile was evaluated. A Human Clariom™ S Pico Assay HT (Applied Biosystems, ThermoFisher Scientific) on a GeneTitan MC Instrument (Applied Biosystems, ThermoFisher Scientific), following the manufacturer's instructions was employed. CEL Intensity files were generated by Affymetrix GeneChip Command Console Software (AGCC, ThermoFisher Scientific). Data analyses were performed using Transcriptomic Analysis Console Software (TAC, ThermoFisher Scientific) that provides quality control analysis, performs normalization and summarization, based on the Signal Space Transformation-Robust Multi-Chip Analysis (SST-RMA) analysis algorithm, and provides a list of differentially expressed genes (Limma Bioconductor package). This phase allows us to obtain a list of differentially expressed genes (DEGs), identified based on their expression fold changes with respect to a relevant control experimental condition (i.e., tumour mass without treatments).

### 7.1.3 IPA Analysis of Transcriptional Profile (Computer Implemented) (Step 1.4)

The use of IPA allows us to estimate how and to what extent the modulation of gene expression in a biological system influences the biological activities related to the pathology of interest.

The transcriptional modifications profile thus obtained is subjected to a functional pathway enrichment analysis. One of the commercial tools that can be used is Ingenuity Pathway Analysis (IPA version 94302991, Qiagen) [Krämer A, et al. Causal analysis approaches in Ingenuity Pathway Analysis. Bioinformatics. 2014]. The list of Differentially expressed genes and corresponding data measurement values (fold change with respect to “Not treated tumour mass) identified in the different experimental conditions were uploaded into the application.

Differentially expressed genes and corresponding fold-changes undergo a filtration process to select only significantly perturbed genes, as indicated by their fold change compared to the “Not treated tumour mass”.

The fold change threshold is set to encompass values≤−2 and ≥+2, accompanied by a statistical significance denoted by a p-value of ≤0.05.

Available identifiers were mapped to their corresponding entity in QIAGEN's Knowledge Base.

By launching “Core Analysis”, significantly perturbed DEGs, called Network Eligible molecules, were overlaid onto a global molecular network developed from information contained in the QIAGEN Knowledge Base. Networks of Network Eligible Molecules were then algorithmically generated based on their connectivity.

The core analysis provides a comprehensive list of approximately top 500 biological activities derived from the generated networks. The associations biological activities-genes are always supported by annotations corresponding to scientific peer review publications that substantiate, through the automatic association of a z-score [Krämer et al. (2014)], the calculated directionality and magnitude of modulation of the biological activities. In essence, this value represents a statistical metric that assesses the similarity between the observed pattern of Differentially Expressed Genes (DEGs) and the expected pattern based on existing literature for a given annotation.

Step 2.1 selection of biological activities that are relevant to the specific pathology under investigation. The selection of biological activities was structured based on the identified hallmarks of the pathology of interest, applying a Z-score threshold set to include values≤−2 and ≥+2, accompanied by a statistical significance denoted by a p-value of ≤0.05.

Step 2.2 The associated Z-score values (FIG. 4 (values in column cluster a)) were used to indicate the directionality and magnitude of modulation for each biological function, the values were converted in absolute values and summed thereby providing the benefit score (step 2.3).

### 7.2 Risk Score Calculation

### 7.2.1 Definition of Side Effects of Cisplatin (Step 3.1)

Officially recorded side effects of cisplatin were identified by consulting specific sources:
torrinomedica.it/schede-farmaci/cisplatino-3/(document made available by AIFA on Jul. 13, 2021)
drugs.com/sfx/cisplatin-side-effects.html as last updated on Mar. 17, 2024
   7.2.2 Identification of IPA Biofunctions Browsing them from the Terms Used to Annotate the Side Effects (Step 3.2)

The information found in the aforementioned resources is used to identify biological activities (The used IPA source is the Ingenuity Knowledge Base, including curation from journal articles, OMIM, JAX and ClinicalTrials.gov) related to the identified side effects and to interrogate IPA through the following procedure:

Side effects terms are written, one by one, in the “disease and functions” query box and the search is then launched.

The obtained resuming table allowed to filter disease/function that come from many lines of evidence (as shown in FIG. 3 where it is possible to appreciate the correspondences between the IPA biological activities and the annotated side effects). The creation of an in-silico model was performed in order to each biological function to a defined number of genes whose modulation is able to influence the modulation of the biological function itself (in this specific case, able to represent the side effect).

7.2.3 Overlay Analysis and Building of the in-Silico Model (Step 3.3)

Overlay analysis focuses on the biological activities identified by the “in-silico model of side effects”. The selection of biological activities is structured based on the identified side effects of cisplatin.

The “Overlay analysis” is structured by establishing relationship between patterns of differentially expressed genes and selected biological activities (always supported by annotations corresponding to scientific peer review publications that substantiate the directionality and magnitude of modulation of the biological activities) using the following procedure:

Import the set of biological activities selected from the in-silico model of the SIDE EFFECT PROFILE of cisplatin into a new sheet called “my pathway.”

Utilize the “Build tool” and “Grow tool” to identify Differentially Expressed Genes (DEGs) belonging to the transcriptomic profile under investigation (cisplatin and EpigenAU/11) and are linked to regulation of the biological activities selected in the previous step.

### 7.2.4 Image Analysis of the Obtained Colour Intensity of Biological Activities (3.4)

Modulation of the identified DEGs is represented using green colour (indicating down-modulation) and red colour (indicating up-modulation).

To determine the expected calculated impact of such experimentally observed modulations of gene expression on biological activities activity, the “Overlay” and “Molecule Activity Predictor” tool (MAP) are employed. The “Prediction” function is activated within MAP tool to calculate the resulting expected modulation of biological activities. Colour coding is thus established:
orange: increase in activity
blue: decrease in activity
white: not achievable/not predictable

The “Overlay analysis” does not directly calculate the Z-score for each biological function. Therefore, it is necessary to translate the colour intensity of the modulation signal into a numerical value. This is achieved by converting the use of dedicated app: “IPAmap_Parser” (version 2.1-1).

The app is a web port app of Pipeline Pilot designed to assign a score, called a z-score, to genes and biofunctions based on their colouring within a biological pathway generated by QIAGEN's Ingenuity Pathway Analysis software. The key step of the algorithm is the conversion from the RGB colour model to the LAB [xrite.com/it-it/blog/lab-color-space] model, a colorimetric encoding that allows for the recording of colour intensity, not just the RGB composition. This conversion takes place within a Pipeline Pilot “component” that utilizes a procedure written in R software, relying on specific functionalities of the colorspace package [cran.r-project.org/web/packages/colorspace/index].

FIG. 33A-B shows final list of biological activities and corresponding values obtained for each treatment under investigation.

### 7.2.5 Calculation of Global Risk Score (Step 3.5)

Risk score is therefore calculated by sum of each positive values obtained from overlay analysis (to indicate that the given side effect is induced by the treatment).

Benefit scores calculation of EpigenAU/11 and cisplatin treatments after 6 hours of treatment (FIG. 33C shows the risk scores obtained by summing each annotated biological activity value for each of the treatments under investigation).

Benefit/risk score calculation was performed by dividing the benefit score obtained by the risk score obtained (see FIG. 6A and B).

The benefit/risk score of product A was calculated with additional steps (introduction of a numeric coefficient of importance of hallmarks)

### 7.3 Benefit Score Calculation

### 7.3.1 CoreE Analysis Biofunctions Selection and Export

Using core analysis obtained at 7.1.3, biological activities taking into account those whose trend is consistent with the therapeutic indications of the product under investigation (i.e. antitumoral activities) were selected. The biological activities were be grouped in activity hallmarks and each hallmark was given a weight coefficient based on its importance in the pathogenesis of interest. Hence biological activities Z-score values were transformed in absolute values and the lists thus obtained are exported in a table which reports the specification of the biofunctions and their relative modulation values. In this, according to step 2.1a case biological activity values were multiplied by the corresponding numeric coefficient of importance, thereby obtaining a final list value that was used for the final calculation of the benefit score (FIG. 4 (values in column cluster b))

The sum of each value for each treatment provided the benefit score (FIG. 6).

Risk score was assessed as previously indicated.

### 7.4 Benefit/Risk Score Calculation

Once the risk scores and the benefit scores have been calculated, they are applied to the following formula:

$$\frac{BENEFIT SCORE}{RISK SCORE}$$

### (See FIG. 6 C and D).

The value of the ratio must therefore be interpreted as follows: the higher the value obtained, the greater the safety of the administered treatment, as the benefits are greater than the risks.

FIG. 6 summarises the results of benefit/risk scores obtained with EpigenAU/11 and cisplatin treatments after 6 hours (A). Calculation of the fold change of the score obtained with EpigenAU/11 compared to that obtained with cisplatin: the EpigenAU/11 score is twice that of cisplatin (B).

When the benefit/risk score is calculated while taking into account the optional steps described above (numeric coefficient of importance), the results obtained are shown in FIG. 6 C and calculation of the fold change of the score obtained with EpigenAU/11 compared to that obtained with cisplatin: the EpigenAU/11 score is still twice that of cisplatin (FIG. 6 D).

Hence, the benefit/risk scores, calculated with and without optional steps, are consistent with each other.

8. Validation of the Proposed Transcriptomics-Based “Method A” Via the Comparison with “Method B”, Intended as Canonical Evaluation of Benefit/Risk Ratio Profile Associated to In Vivo Administration of EpigenAU/11 And Cisplatin

### 8.1. In Vivo Assay (Step 1)

EpigenAU/11 (DoE2) and cisplatin were administered in vivo in a mouse model. The risk (intended as side effects) was estimated through the observation of behavioural parameters (impacting the behaviour itself, the locomotor system, muscle strength and nervous reflexes) and through the potential weight loss of the animal.

8.2 EpigenAU/11 and Cisplatin Benefit/Risk Ratio Comparison Based on Data Obtained from In Vivo Experimentation

### 8.2.1 Risk Score Calculation

Data obtained by animal testing, considered in the context of the risk, are behavioural parameters and body weight loss at the end of experimentation.

The behavioural parameters monitored are listed below:
1. Loss of Spontaneous activity
2. Loss of Cleaning
3. Loss of Curiosity
4. Loss of Reactivity
5. Loss of Straightening reflex
6. Loss of Physical strength
7. Impairment of Palpebral opening
8. Palpebral reflex
9. Tremors
10. Pallor
11. Stereotypies
12. Passivity

The values of the parameters 1-8 are normalized as: “Not treated animal value” (always equal to 0)+“Treated animal value”.

The values of the parameters 9-12 are normalized as: “Not treated animal value”−“Treated animal value”.

The potential weight loss of the treated animals was also assessed.” Data were processed using the formula:

$$1 - [ “ Body weight of treated animals ( day of observation : 27 ) / body weight of untreated animals ( day of observation : 19 ) ]$$

The table below summarises the Risk score calculation of EpigenAU/11 and cisplatin treatments with sum of the data derived from in vivo experimentation.

The table below shows the experimental values, representing the in vivo side effects, that have not yet been reprocessed for the purpose of risk score calculation.

| | | RISK | Vehicle | | EpigenAU/11 | | Cisplatin | | |
|---|---|---|---|---|---|---|---|---|---|
| | HALLMARK | PARAMETERS | Mean | Dev. st | Mean | Dev. st | Mean | Dev. st | Limits |
| BEHAVIOURAL | Behaviour | Spontaneous activity | 4.00 | 0.00 | 3.70 | 0.45 | 2.50 | 1.58 | 4-0 |
| DATA | | Passivity | 0.00 | 0.00 | 0.30 | 0.45 | 1.60 | 1.52 | 0-4 |
| | | Cleaning | 4.00 | 0.00 | 3.80 | 0.45 | 2.90 | 1.67 | 4-0 |
| | | Curiosity | 4.00 | 0.00 | 3.80 | 0.27 | 2.60 | 1.67 | 4-0 |
| | | Reactivity | 4.00 | 0.00 | 3.90 | 0.22 | 2.20 | 1.30 | 4-0 |
| | Locomotor system | Stereolipies | 0.00 | 0.00 | 0.00 | 0.00 | 0.00 | 0.00 | 0-4 |
| | | Straightening reflex | 4.00 | 0.00 | 3.80 | 0.45 | 2.60 | 1.47 | 4-0 |
| | Muscle | Physical strenght | 4.00 | 0.00 | 3.80 | 0.27 | 2.70 | 1.68 | 4-0 |
| | Nervous system | Palpebral reflex | 4.00 | 0.00 | 3.80 | 0.45 | 2.80 | 1.68 | 4-0 |
| | | Pallor | 0.00 | 0.00 | 1.20 | 0.84 | 2.00 | 1.22 | 0-4 |
| | | Palpebral opening | 4.00 | 0.00 | 3.50 | 0.71 | 2.00 | 0.94 | 4-0 |
| | | Tremors | 0.00 | 0.00 | 0.10 | 0.22 | 0.80 | 1.10 | 0-4 |
| BODY WEIGHT | Nutritional and | Body weight | 37.48 | 1.89 | 35.26 | 1.60 | 29.12 | 2.93 | / |
| DATA | metabolic system | | | | | | | | |

### 8.3 Benefit Score Calculation (Referring to Method B Step 2)

Data obtained by animal testing, considered in the context of benefit, is the tumour mass size reached at the end of in vivo test for each treatment.

Data were calculated as “Reduction in the size of tumour mass” by the formula:

1−[“Tumour mass size collected from treated animals (day of observation: 27)/Tumour mass size collected from untreated animals (day of observation: 19)]

Below is reported the benefit score calculation of EpigenAU/11 and cisplatin treatments with data derived from in vivo experimentation.

The benefit (intended as therapeutic activity) was instead evaluated by taking into consideration the reduction in size of the tumour mass compared to the untreated tumour. For this purpose, immunocompromised mice bearing xenografted FaDu head and neck squamous carcinoma cells were included in the study once tumours reached a volume of 100 mm³. Mice received daily intratumoral injections of EpigenAU/11 and intraperitoneal cisplatin on alternating days.

The table below shows the experimental values, representing the in vivo benefit effect, that have not yet been reprocessed for the purpose of benefit score calculation.

| | BENEFIT | Vehicle | | EpigenAU/11 | | Cisplatin | |
|---|---|---|---|---|---|---|---|
| HALLMARK | PARAMETERS | Mean | Dev. st | Mean | Dev. st | Mean | Dev. st |
| Tumor viability | Size of tumor masses | 1.38 | 0.11 | 0.15 | 0.12 | 0.50 | 0.35 |

The values obtained show that the canonically evaluated benefit/risk ratio profile associated to EpigenAU/11 is more favourable with respect to that associated to cisplatin. Data generated using a canonical in vivo observation thus confirms the predictability of the hereby reported novel method based on ex vivo transcriptomics and substantiates its validation.

Alternatively, data previously and separately obtained from in vivo experiments can be used for the method of the invention.

### 8.4 Benefit/Risk Score Calculation

Once the risk scores and the benefit scores have been calculated, they are applied to the following formula:

$$\frac{BENEFIT SCORE}{RISK SCORE}$$

The value of the ratio must therefore be interpreted as: the higher the value obtained, the greater the safety of the administered treatment, as the benefits are greater than the risks. FIG. 7 shows the Benefit/Risk scores obtained with in vivo EpigenAU/11 and cisplatin treatments (A). Calculation of the fold change of the score obtained with EpigenAU/11 compared to that obtained with cisplatin: the EpigenAU/11 score is three times that of cisplatin (B).

The two methods disclosed in examples 7 and 8 (with or without optional steps) proved to be useful in order to objectivate the comparison between benefit/risk profile of two anticancer treatments. The first method is based on ex vivo transcriptomics data and proved to be able to correctly predict the favourability of EpigenAU/11 profile with respect to that of cisplatin, undergoing a validation based on comparison with results generated using a canonical qualitative approach based on in vivo data. The second method enables to summarize such in vivo data in a single parameter that enables that facilitates and renders more easily approachable the comparison between the benefit/risk profiles of therapeutic options. Both methods produce similar results demonstrating the superiority of EpigenAU/11's benefit/risk score over Cisplatin (FIG. 36), thus proving the consistency of the ex vivo method with the one generated using a canonical in vivo approach, thereby representing a favourable profile for EpigenAU/11 with respect to that of Cisplatin, returning a result coherent with that generated using a canonical in vivo approach.

### 9. New Benefit/Risk Assessment Product B (Computer Implemented/Assisted)

### 9.1 Data Assembly

### 9.1.1 In Vitro Sample Treatment and RNA Extraction (Steps 1.1 and 1.2)

Human, adipocyte-derived, mesenchymal stem cell lines (hADMSC), capable of differentiating into osteoblasts and mineralize the extracellular matrix (ECM) were used in this study. These cells were obtained during general surgery from three different patients (PA42, PA59 and PA69) (Romagnoli et al, “In Vitro Behavior of Human Adipose Tissue-Derived Stem Cells on Poly(ε-caprolactone) Film for Bone Tissue Engineering Applications”, BioMed Research International, vol. 2015, Article ID 323571, 12 pages, 2015.doi/10.1155/2015/323571). These cell lines have been characterized with respect to the main stemness markers of mesenchymal stem cells (CD44, CD105, and STRO1) and by studying their multipotency toward osteogenic phenotypes at the Department of Surgery and Translational medicine of the University of Florence.

hADMSCs were cultured in a growth medium (GM) and grown to 70-80% confluence. Afterwards, the cells were seeded in 24-well plate at a concentration of 1×105 cells/well. After a week, the GM was replaced with osteogenic medium (OM) containing the fluorophore calcein 1 μg/mL and incubated for 28 days with or without Osteoredux or DiBase. The medium with or without the product C was refreshed twice a week.

### 9.1.2 Transcriptome Raw Data Analysis (Step 1.3)

Whole transcriptome expression profile was evaluated. A Human Clariom™ S Pico Assay HT (Applied Biosystems, ThermoFisher Scientific) on a GeneTitan MC Instrument (Applied Biosystems, ThermoFisher Scientific), following the manufacturer's instructions was employed. CEL Intensity files were generated by Affymetrix GeneChip Command Console Software (AGCC, ThermoFisher Scientific). Data analyses were performed using Transcriptomic Analysis Console Software (TAC, ThermoFisher Scientific) that provides quality control analysis, performs normalization and summarization, based on the Signal Space Transformation-Robust Multi-Chip Analysis (SST-RMA) analysis algorithm, and provides a list of differentially expressed genes (Limma Bioconductor package). This phase allows us to obtain a list of differentially expressed genes (DEGs).

### 9.1.3 IPA Analysis of Transcriptional Profile (Computer Implemented) (Step 1.4)

The use of IPA allows us to estimate how and to what extent the modulation of gene expression in a biological system influences the biological activities related to the pathology of interest.

The transcriptional modifications profile thus obtained is subjected to a functional pathway enrichment analysis. One of the commercial tools that can be used is Ingenuity Pathway Analysis (IPA version 94302991, Qiagen) [Krämer A, et al. Causal analysis approaches in Ingenuity Pathway Analysis. Bioinformatics. 2014]. The list of Differentially expressed genes and corresponding data measurement values identified in the different experimental conditions were uploaded into the application.

Differentially expressed genes and corresponding fold-changes undergo a filtration process to select only significantly perturbed genes, as indicated by their fold change compared to the positive control.

The fold change threshold is set to encompass values≤−1.5 and ≥+1.5, accompanied by a statistical significance denoted by a p-value of ≤0.05.

Available identifiers were mapped to their corresponding entity in QIAGEN's Knowledge Base.

By launching “Core Analysis”, significantly perturbed DEGs, called Network Eligible molecules, were overlaid onto a global molecular network developed from information contained in the QIAGEN Knowledge Base. Networks of Network Eligible Molecules were then algorithmically generated based on their connectivity.

The core analysis provides a comprehensive list of approximately top 500 biological activities derived from the generated networks. The associations biological activities-genes are always supported by annotations corresponding to scientific peer review publications that substantiate, through the automatic association of a z-score [Krämer et al. (2014)], the calculated directionality and magnitude of modulation of the biological activities. In essence, this value represents a statistical metric that assesses the similarity between the observed pattern of Differentially Expressed Genes (DEGs) and the expected pattern based on existing literature for a given annotation.

Step 2.1 selection of biological activities that are relevant to the specific pathology under investigation. The selection of biological activities was structured based on the identified hallmarks of the pathology of interest, applying a fold-change threshold set to include values≤−1.5 and ≥+1.5, accompanied by a statistical significance denoted by a p-value of ≤0.05. Step 2.2 The associated Z-score values (FIG. 9 (values in column cluster a)) were used to indicate the directionality and magnitude of modulation for each biological function, the values were converted in absolute values and summed thereby providing the benefit score (step 2.3).

### 9.2 Risk Score Calculation

### 9.2.1 Definition of Side Effects of DIBASE (Step 3.1)

Officially recorded side effects of DIBASE were identified by consulting specific sources:
torrinomedica.it/schede-farmaci/dibase/(document made available by AIFA on Jul. 13, 2021)
drugs.com/sfx/cholecalciferol-side-effects.html as last updated on Mar. 17, 2024
   9.2.2 Identification of IPA Biofunctions Browsing them from the Terms Used to Annotate the Side Effects (Step 3.2)

The information found in the aforementioned resources is used to identify biological activities (The used IPA source is the Ingenuity Knowledge Base, including curation from journal articles, OMIM, JAX and ClinicalTrials.gov) related to the identified side effects and to interrogate IPA through the following procedure:

Side effects terms are written, one by one, in the “disease and functions” query box and the search is then launched.

The obtained resuming table allowed to filter disease/function that come from many lines of evidence (as shown in FIG. 8 where it is possible to appreciate the correspondences between the IPA biological activities and the annotated side effects). The creation of an in-silico model was performed in order to each biological function to a defined number of genes whose modulation is able to influence the modulation of the biological function itself (in this specific case, able to represent the side effect).

9.2.3 Overlay Analysis and Building of the in-Silico Model (Step 3.3)

Overlay analysis focuses on the biological activities identified by the “in-silico model of side effects”. The selection of biological activities is structured based on the identified side effects of DIBASE.

The “Overlay analysis” is structured by establishing relationship between patterns of differentially expressed genes and selected biological activities (always supported by annotations corresponding to scientific peer review publications that substantiate the directionality and magnitude of modulation of the biological activities) using the following procedure:

Import the set of biological activities selected from the in-silico model of the SIDE EFFECT PROFILE of DIBASE into a new sheet called “my pathway.”

Utilize the “Build tool” and “Grow tool” to identify Differentially Expressed Genes (DEGs) belonging to the transcriptomic profile under investigation (DIBASE and Osteoredux) and are linked to regulation of the biological activities selected in the previous step.

### 9.2.4 Image Analysis of the Obtained Colour Intensity of Biological Activities (3.4)

Modulation of the identified DEGs is represented using green colour (indicating down-modulation) and red colour (indicating up-modulation).

To determine the expected calculated impact of such experimentally observed modulations of gene expression on biological activities activity, the “Overlay” and “Molecule Activity Predictor” tool (MAP) are employed. The “Prediction” function is activated within MAP tool to calculate the resulting expected modulation of biological activities. Colour coding is thus established:
orange: increase in activity
blue: decrease in activity
white: not achievable/not predictable

The “Overlay analysis” does not directly calculate the Z-score for each biological function. Therefore, it is necessary to translate the colour intensity of the modulation signal into a numerical value. This is achieved by converting the use of dedicated app: “IPAmap_Parser” (version 2.1-1).

The app is a web port app of Pipeline Pilot designed to assign a score, called a z-score, to genes and biofunctions based on their colouring within a biological pathway generated by QIAGEN's Ingenuity Pathway Analysis software. The key step of the algorithm is the conversion from the RGB colour model to the LAB [xrite.com/it-it/blog/lab-color-space] model, a colorimetric encoding that allows for the recording of colour intensity, not just the RGB composition. This conversion takes place within a Pipeline Pilot “component” that utilizes a procedure written in R software, relying on specific functionalities of the colorspace package [cran.r-project.org/web/packages/colorspace/index].

FIG. 9 shows final list of biological activities and corresponding values obtained for each treatment under investigation.

### 9.2.5 Calculation of Global Risk Score (Step 3.5)

Risk score is therefore calculated by sum of each positive values obtained from overlay analysis (to indicate that the given side effect is induced by the treatment). Benefit scores calculation of Osteoredux and DIBASE treatments (FIG. 10 shows the risk scores obtained by summing each annotated biological activity value for each of the treatments under investigation).

Benefit/risk score calculation was performed by dividing the benefit score obtained by the risk score obtained (see FIG. 11A and B).

The benefit/risk score of product B was calculated with additional steps (introduction of a numeric coefficient of importance of hallmarks)

9.3 Benefit score calculation
9.3.1 Core analysis biofunctions selection and export

Using core analysis obtained at 7.1.3, biological activities taking into account those whose trend is consistent with the therapeutic indications of the product under investigation (i.e., remodelling of bone) were selected. The biological activities were be grouped in activity hallmarks and each hallmark was given a weight coefficient based on its importance in the pathogenesis of interest. Hence biological activities Z-score values were transformed in absolute values and the lists thus obtained are exported in a table which reports the specification of the biofunctions and their relative modulation values. In this, according to step 2.1a case biological activity values were multiplied by the corresponding numeric coefficient of importance, thereby obtaining a final list value that was used for the final calculation of the benefit score (FIG. 9 (values in column cluster b))

The sum of each value for each treatment provided the benefit score (FIG. 9). Risk score was assessed as previously indicated.

### 9.4 Benefit/Risk Score Calculation

Once the risk scores and the benefit scores have been calculated, they are applied to the following formula:

$$\frac{BENEFIT SCORE}{RISK SCORE}$$

### (See FIG. 11 C and D).

The value of the ratio must therefore be interpreted as follows: the higher the value obtained, the greater the safety of the administered treatment, as the benefits are greater than the risks.

FIG. 11 summarises the results of benefit/risk scores obtained with Osteoredux and DIBASE treatments (A). Calculation of the fold change of the score obtained with Osteoredux compared to that obtained with DIBASE: the Osteoredux score is three times that of DIBASE (B).

When the benefit/risk score is calculated while taking into account the optional steps described above (numeric coefficient of importance), the results obtained are shown in FIG. 11 C and calculation of the fold change of the score obtained with Osteoredux compared to that obtained with DIBASE: the Osteoredux score is six times that of DIBASE (FIG. 11 D).

Hence, the benefit/risk scores, calculated with and without optional steps, are consistent with each other.

1.-17. (canceled)

18. A computer-implemented method for providing a benefit/risk score for a therapeutic product of interest used to treat a pathology or pathological state, said method comprising:
(1) performing a transcriptomic data analysis comprising:
(1.1) providing one or more samples of a biological substrate representing the pathology or pathological state treated by said therapeutic product;
(1.2) contacting said one or more of samples with said therapeutic product or a reference drug known to treat said pathology or pathological state;
(1.3) extracting ribonucleic acid (RNA) from each of said samples in 1.1 and 1.2;
(1.4) identifying differentially expressed genes (DEGs) in each of samples 1.2 with respect to said samples 1.1, and determining the changes in gene expression for each DEG relative to the samples in 1.1, thereby obtaining fold change values for each of said DEG; and
(1.5) determining, by use of a data processor, numerical values representing the variation of gene expression in 1.4 in terms of magnitude and directionality of one or more biological activities related to the differential expression of said DEGs by pathway enrichment and functional analysis;
(2) determining a benefit score comprising:
(2.1) selecting one of more biological activities from 1.5 that are associated with said pathology or pathological state to be treated by the therapeutic product of interest;
(2.2) converting the numerical values of 1.5, by use of a data processor, into absolute values; and
(2.3) summing, by use of a data processor, said absolute values of each of the one or more biological activities from 2.2 thereby obtaining a benefit score for the therapeutic product of interest;
(3) determining a risk score comprising:
(3.1) providing a listing of side effects associated with the reference drug of 1.2;
(3.2) identifying determining one or more biological activities associated with said side effects;
(3.3) identifying a relationship between the gene expression in 1.4 and said one or more biological activities in 3.2 using an in-silico model of said pathology or pathological state by pathway and functional analysis;
(3.4) identifying the degree of regulation of each of said one or more biological activity in 3.2 by inputting, in a data processor, the fold change value for each of said DEG in 1.4 to said in-silico model using pathway and functional analysis, and transforming said degree of regulation into absolute numerical values; and
(3.5) obtaining a risk score of the therapeutic product of interest by summing, through use of a data processor, the absolute numerical values in 3.4;
(4) providing a benefit/risk score for the therapeutic product of interest as the ratio of the benefit score value obtained in 2.3 and the risk score value obtained in 3.5.

19. The method of claim 18, wherein the risk score of 3.4 is adjusted to a predetermined positive minimal risk score value if it is less than said predetermined minimal risk score value.

20. (canceled)

21. The method of claim 20, wherein the bioinformatics analysis is performed using QIAGEN Ingenuity Pathway Analysis (IPA)®, MetaCore (Clarivate Analytics)®, GeneGo (by MetaCore), Qiagen OmicSoft, Elsevier Pathway Studio®, Ingenuity Variant Analysis (IVA, Qiagen), DAVID (Database for Annotation, Visualization, and Integrated Discovery), GSEA (Gene Set Enrichment Analysis), STRING (Search Tool for the Retrieval of Interacting Genes/Proteins), KEGG Pathway Analysis®, or Metascape, Cytoscape® with enrichment plugins.

22. The method of claim 18, wherein said fold changes values obtained in 1.4 are expressed as Z-score values.

23. The method of claim 18, wherein 2.1 further comprises clustering of said biological activities into hallmarks of the pathology or pathological state treated by the therapeutic product of interest and providing a numerical coefficient of importance to each of said hallmarks; and 2.2 further comprises multiplying each of the numerical values obtained in 1.5 of said one or more biological activities by said numerical coefficient of the hallmark in which said one or more biological activities is clustered prior to converting said numerical values into absolute values.

24. The method of claim 23, wherein each of said numerical coefficient of importance is determined based on a frequency of association, statistical relevance, or machine learning-based feature importance.

25. The method of claim 18, wherein 3.2 is performed using a machine learning model trained with transcriptomic datasets.

26. The method of claim 23, wherein clustering of said biological activities into hallmarks of the pathology or pathological state treated by the therapeutic product of interest and providing a numerical coefficient of importance to each of said hallmarks is performed using a machine learning model trained with transcriptomic datasets.

27. The method of claim 18, further comprising: assigning a numerical coefficient of importance to each side effect determined in step 3.1 and multiplying the fold change values obtained in step 1.4, which are associated with each side effect, by their corresponding numerical coefficients of importance.

28. The method of claim 27, wherein each of said numerical coefficient of importance is calculated based on one or more of severity of the side effect according to clinical grading systems, impact on patient-reported quality of life, and duration of the side effect.

29. The method of claim 18, further comprising performing steps 1-4 on a reference drug known for the treatment of said pathology or pathological state, thereby providing a benefit/risk score value for said reference drug.

30. The method of claim 18, that further comprises the steps of determining a benefit/risk score based on data previously obtained from in vivo animal models, wherein:
(a) the biological samples are obtained from animal models representing the pathology or pathological states that were: (a1) treated with said therapeutic product of interest or (a2) a reference drug known to treat said pathology or pathological state; or (a3) a control sample; and
(b) identifying the numerical values of 1.5 wherein the biological activities are behavioral modifications and/or body weight-loss indicative of animal suffering from said pathology or pathological state, and therapeutic efficacy in each of groups (a1) and (a2) normalized against control group (a3).

31. The method of claim 30, further comprising performing steps 1-4 on the reference drug known to treat said pathology or pathological state, thereby providing a benefit/risk score value for said reference drug.

32. A method for selecting a therapeutic or beneficial product useful to treat a pathology or pathological state for clinical development comprising calculating the benefit/risk score value for said product by performing the method of claim 18 and selecting a therapeutic or beneficial product for clinical development when the benefit/risk score value is equal to or greater than said benefit/risk score value calculated for the reference drug known to treat the pathology or pathological state.

33. The method of claim 32, that further comprises the steps of determining benefit/risk score value for said therapeutic or beneficial product and the reference drug by:
(a) obtaining the biological samples from animal models representing the pathology or pathological states that were: (a1) treated with said therapeutic product of interest or (a2) a reference drug known to treat said pathology or pathological state; or (a3) a control sample; and
(b) identifying the numerical values of 1.5 wherein the biological activities are behavioral modifications and/or body weight-loss indicative of animal suffering from said pathology or pathological state, and therapeutic efficacy in each of groups (a1) and (a2) normalized against control group (a3).

34. The method of claim 18, wherein said therapeutic product of interest comprises said one or more natural matrices.

35. The method of claim 34, wherein said natural matrices are obtained from plant, marine, bacterial, fungi, yeasts, animal, or other natural sources.

36. The method of claim 35, wherein said natural matrices are selected from cut or pulverized plant parts, plant extracts, fractions of said extracts, microorganisms, honey, propolis, silk, wax, plant resins, plant gums, plant exudates, vegetable oils, vegetable essential oils, animal tissues lysates, or plant or animal fluids.
